(19) United States
(12) Patent Application Publication
(10) Pub. No.: US 2005/0187545 A1
(43) Pub. Date: Aug. 25, 2005

(54) Magnetic catheter ablation device and method

(76) Inventors: Michael D. Hooven, Cincinnati, OH (US); David W. Rister, Cincinnati, OH (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/059,954

(22) Filed: Feb. 17, 2005

Related U.S. Application Data
(60) Provisional application No. 60/546,138, filed on Feb. 20, 2004.

(51) Int. Cl. A61B 18/18

(52) U.S. Cl. 606/41; 606/21

(57) ABSTRACT

A method and apparatus for ablation of a layer of tissue is achieved by providing first and second bodies on opposed sides of the tissue. The first body includes a first ablation member and a source of magnetic force adjacent one side of the tissue. The second body includes a second ablation member and a magnetically attractive element responsive to the magnetic force adjacent the other side of the tissue. The magnetic attraction between the source and the attractive element is adapted to align the first and second bodies in opposed relationship on the opposed sides of the tissue. One of the first and second bodies may include at least one expandible member for controlling the magnetic attraction between the bodies.

### BACKGROUND OF THE INVENTION

This application is a non-provisional application which claims the benefit of provisional application Ser. No. 60/546,138, filed Feb. 20, 2004, which application is incorporated by reference herein.

Atrial fibrillation is the most common heart arrhythmia in the world, affecting over 2.5 million people in the United States alone. Ablation of cardiac tissue, in order to create scar tissue that poses an interruption in the path of the errant electrical impulses in the heart tissue, is a commonly performed procedure to treat cardiac arrhythmias. Such ablation may range from the ablation of a small area of heart tissue to a series of ablations forming a strategic placement of incisions in both atria to stop the conduction and formation of errant impulses.

Ablation has been achieved or suggested using a variety of techniques, such as freezing via cryogenic probe, heating via RF energy, surgical cutting and other techniques. As used here, “ablation” means the removal or destruction of the function of a body part, such as cardiac tissue, regardless of the apparatus or process used to carry out the ablation. Also, as used herein, “transmural” means through the wall or thickness, such as through the wall or thickness of a hollow organ or vessel.

Ablation of cardiac tissue may be carried out in an open surgical procedure, where the breastbone is divided and the surgeon has direct access to the heart, or through a minimally invasive route, such as between the ribs, through a sub-xyphoid incision or via catheter that is introduced through a vein, and into the heart.

Prior to any ablation, the heart typically is electronically mapped to locate the point or points of tissue which are causing the arrhythmia. With minimally invasive procedures such as via a catheter, the catheter is directed to the aberrant tissue, and an electrode or cryogenic probe is placed in contact with the endocardial tissue. RF energy is delivered from the electrode to the tissue to heat and ablate the tissue (or the tissue may be frozen by the cryogenic probe), thus eliminating the source of the arrhythmia.

Common problems encountered in this procedure are difficulty in precisely locating the aberrant tissue, and complications related to the ablation of the tissue. Locating the area of tissue causing the arrhythmia often involves several hours of electrically “mapping” the inner surface of the heart using a variety of mapping catheters, and once the aberrant tissue is located, it is often difficult to position the catheter and the associated electrode or probe so that it is in contact with the desired tissue.

The application of either RF energy or ultra-low temperature freezing to the inside of the heart chamber also carries several risks and difficulties. It is very difficult to determine how much of the catheter electrode or cryogenic probe surface is in contact with the tissue since catheter electrodes and probes are cylindrical and the heart tissue cannot be visualized clearly with existing fluoroscopic technology. Further, because of the cylindrical shape, some of the exposed electrode or probe area will almost always be in contact with blood circulating in the heart, giving rise to a risk of clot formation.

Clot formation is almost always associated with RF energy or cryogenic delivery inside the heart because it is difficult to prevent the blood from being exposed to the electrode or probe surface. Some of the RF current flows through the blood between the electrode and the heart tissue and this blood is coagulated, or frozen when a cryogenic probe is used, possibly resulting in clot formation. When RF energy is applied, the temperature of the electrode is typically monitored so as to not exceed a preset level, but temperatures necessary to achieve tissue ablation almost always result in blood coagulum forming on the electrode.

Overheating or overcooling of tissue is also a major complication, because the temperature monitoring only gives the temperature of the electrode or probe, which is, respectively, being cooled or warmed on the outside by blood flow. The actual temperature of the tissue being ablated by the electrode or probe is usually considerably higher or lower than the electrode or probe temperature, and this can result in overheating, or even charring, of the tissue in the case of an RF electrode, or freezing of too much tissue by a cryogenic probe. Overheated or charred tissue can act as a locus for thrombus and clot formation, and over freezing can destroy more tissue than necessary.

It is also very difficult to achieve ablation of tissue deep within the heart wall. A recent study reported that to achieve a depth of ablation of 5 mm, it was necessary to ablate an area almost 8 mm wide in the endocardium. See, “Mechanism, Localization, and Cure of Atrial Arrhythmias Occurring After a New Intraoperative Endocardial Radiofrequency Ablation Procedure for Atrial Fibrillation,” Thomas, et al., J. Am. Coll. Cardiology, Vol. 35, No. 2, 2000. As the depth of penetration increases, the time, power, and temperature requirements increase, thus increasing the risk of thrombus formation.

In certain applications, it is desired to obtain a continuous line of ablated tissue in the endocardium. Using a discrete or point electrode or probe, the catheter must be “dragged” from point to point to create a line, and frequently the line is not continuous. Multielectrode catheters have been developed which can be left in place, but continuity can still be difficult to achieve because of the difficulty in maintaining good tissue contact, and the lesions created can be quite wide.

Because of the risks of char and thrombus formation, RF energy, or any form of endocardial ablation, is rarely used on the left side of the heart, where a clot could cause a serious problem (e.g., stroke). Because of the physiology of the heart, it is also difficult to access certain areas of the left atrium via an endocardial, catheter-based approach.

Recently, epicardial ablation devices have been developed which apply RF energy to the outer wall of the heart to ablate tissue. These devices do not have the same risks concerning thrombus formation. However, it is still difficult to create long, continuous lesions, and it is difficult to achieve good depth of penetration without creating a large area of ablated tissue.

As noted above, other forms of energy have been used in ablation procedures, including ultrasound, cryogenic ablation, laser, and microwave technology. When used from an endocardial approach, the limitations of all energy-based ablation technologies to date are the difficulty in achieving continuous transmural lesions, and minimizing unnecessary damage to endocardial tissue. Ultrasonic and RF energy endocardial balloon technology has been developed to create circumferential lesions around the individual pulmonary veins. See e.g., U.S. Pat. No. 6,024,740 to Lesh et al. and U.S. Pat. Nos. 5,938,660 and 5,814,028 to Swartz et al. However, this technology creates rather wide (greater than 5 mm) lesions which could lead to stenosis (narrowing) of the pulmonary veins. See, “Pulmonary Vein Stenosis after Catheter Ablation of Atrial Fibrillation,” Robbins, et al., Circulation, Vol. 98, pages 1769-1775, 1998. The large lesion area can also act as a locus point for thrombus formation. Additionally, there is no feedback to determine when full transmural ablation has been achieved. Cryogenic ablation has been attempted both endocardially and epicardially (see e.g., U.S. Pat. No. 5,733,280 to Avitall, U.S. Pat. No. 5,147,355 to Friedman et al., and U.S. Pat. No. 5,423,807 to Milder, and WO 98/17187, the latter disclosing an angled cryogenic probe, one arm of which is inserted into the interior of the heart through an opening in the heart wall that is hemostatically sealed around the arm by means of a suture or staples), but because of the time required to freeze tissue, and the delivery systems used, it is difficult to create a continuous line, and uniform transmurality is difficult to verify.

Published PCT applications WO 99/56644 and WO 99/56648 disclose an endocardial ablation catheter with a reference plate located on the epicardium to act as an indifferent electrode or backplate that is maintained at the reference level of the generator. Current flows either between the electrodes located on the catheter, or between the electrodes and the reference plate. It is important to note that this reference plate is essentially a monopolar reference pad. Consequently, there is no energy delivered at the backplate/tissue interface intended to ablate tissue. Instead, the energy is delivered at the electrode/tissue interface within the endocardium, and travels through the heart tissue either to another endocardial electrode, or to the backplate. Tissue ablation proceeds from the electrodes in contact with the endocardium outward to the epicardium. Other references disclose epicardial multielectrode devices that deliver either monopolar or bipolar energy to the outside surface of the heart.

It is important to note that all endocardial ablation devices that attempt to ablate tissue through the full thickness of the cardiac wall have a risk associated with damaging structures within or on the outer surface of the cardiac wall. As an example, if a catheter is delivering energy from the inside of the atrium to the outside, and a coronary artery, the esophagus, or other critical structure is in contact with the atrial wall, the structure can be damaged by the transfer of energy from within the heart to the structure. The coronary arteries, esophagus, aorta, pulmonary veins, and pulmonary artery are all structures that are in contact with the outer wall of the atrium, and could be damaged by energy transmitted through the atrial wall.

Several devices and methods utilizing ablation in the treatment of atrial fibrillation have been described in co-pending applications to the current inventor: Ser. No. 60/464,713, a provisional application, filed Apr. 23, 2003, Ser. No. 60/441,661, a provisional application, filed Jan. 22, 2003, Ser. No. 10/158,985, filed May 31, 2002, which together with Ser. Nos. 10/015,476 and 10/015,440, both filed Dec. 13, 2001, and Ser. Nos. 10/015,303, 10/015,346, 10/015,862, and 10/015,868, all filed Dec. 12, 2001, are all divisional applications of application Ser. No. 10/038,506, filed Nov. 9, 2001, which is a continuation-in-part of application Ser. No. 10/032,378, filed Oct. 26, 2001, which is a continuation-in-part of application Ser. No. 09/844,225 filed Apr. 27, 2001, which is a continuation-in-part of application Ser. No. 09/747,609 Dec. 22, 2000, which claims the benefit of provisional application Ser. No. 60/200,072, filed Apr. 27, 2000. These applications are hereby incorporated by reference in the present application.

Accordingly, it is the object of the present invention to provide an improved method and apparatus for making transmural ablations to heart tissue.

It is a related object to provide a method and apparatus for making transmural ablation at a selected cardiac location that minimizes unnecessary damage to the heart tissue.

It is a further object to provide a method and apparatus for making transmural ablation at a selected cardiac location that employs magnetic attraction to engage and clamp layers of heart tissue at the selected location.

It is also a further object to provide a method and apparatus that creates magnetic attraction between portions of the apparatus which are each disposed on generally opposite sides of a layer of heart tissue and ablates the tissue therebetween.

It is also an object to provide a method and apparatus for guiding an ablation instrument to a selected cardiac location prior to ablation utilizing a guide facility.

It is a yet further object to provide a method and apparatus for guiding an ablation instrument to generally opposite sides of a pericardial reflection by employing a magnetic attraction provided by the apparatus.

It is still a further object to provide a method and apparatus for ablating cardiac tissue which utilizes an expandible member.

### SUMMARY OF THE INVENTION

These objects, and others will become apparent upon reference to the following detailed description and attached drawings are achieved by the use of an apparatus for ablating tissue, preferably cardiac tissue.

In a first embodiment of the invention, the apparatus includes a first elongated body having a distal end, a proximal end and a source of magnetic force. A second elongated body has a distal end, a proximal end and a magnetically attractive element which is responsive to the magnetic force. Each of the source of magnetic force and the magnetically attractive element are preferably carried at the distal end of the body although it is conceivable that they may be disposed at other locations along the length of the body. Each of the source and the magnetically attractive element may be either a temporary magnet having a magnetic field which is created upon energizing an insulated wire coil with an electrical current applied to the wire by a current source and conventionally known as an electromagnet, or, alternatively, a permanent magnet, which is comprised of a plurality of arranged particles which together create a magnet by their arrangement, or, as an even further alternative, a combination of both. It is also possible that the magnetically attractive element may be made of a magnetic material which is attracted by at least a portion of the source, regardless of whether the source is a temporary or permanent magnet or both.

Each body also comprises an ablation member connected to an ablation activation source for ablating tissue therebetween. The magnetic attraction between the first and second bodies facilitates alignment of the bodies on opposed sides of the tissue. The ablation activation source is preferably located outside the patient's body.

The method achieved by the use of the apparatus and includes the steps of providing the first and second body adjacent opposing sides of the tissue which is identified for ablation. The first and second bodies may be inserted into the body cavity and advanced to the tissue by one or more various approaches which will be discussed in more detail below. Preferably, the first body employs a sub-xyphoid or intercostal approach whereby it is inserted under the sub-xyphoid process or between the ribs and advanced to the epicardial surface of the heart and the second body employs an approach through which it is inserted into a femoral or subclavian vein, and follows a path to the heart for ablation of heart tissue.

The magnetic attraction between the source and the magnetically attractive element facilitate appropriate positioning and alignment of the bodies on opposite sides of a layer of cardiac tissue. The ablation members are activated and the tissue is ablated.

The method may be performed using at least one flexibly elongated guide facility which may be inserted into the body cavity so as to further aid in advancing one or more bodies to the selected tissue site. One of the first and second bodies may be attached to the guide facility to draw the respective body to the selected tissue location, or, alternatively, at least one of the first and second bodies may define a channel for receiving at least a portion of the guide facility. The guide facility may be pre-shaped by the operator and guided to the selected tissue location using conventional methods such as, for example, fluoroscopy. Any of the first and second bodies may be a flexible so that the body follows the shape of the guide facility which is received within the channel.

The method may further be performed using at least one expandible member which is preferably but not exclusively a balloon. A first expandible member is located on one of the first and second bodies, preferably the first body which is introduced to an epicardial surface. The first expandible member is preferably located on the distal end in the vicinity of the source of magnetic force. Expansion of the expandible member moves the source of magnetic force away from the epicardial surface and decreases the magnet force acting on the second or endocardially-disposed body to facilitate positioning of the second body. The expandible member is retracted prior to ablation to increase the magnetic attraction and facilitate alignment of the bodies on the opposite sides of the tissue. The expandible member may be re-inflated to decrease the magnetic force to allow for re-positioning of the second body and/or engagement with other selected ablation locations. A second expandible member may be employed on the first body, preferably in opposed relation to the first expandible member, and may be inflated upon deflation of the first expandible member, so as to bias the epicardially-disposed body adjacent the epicardial surface.

### BRIEF DESCRIPTION OF THE DRAWINGS

FIG. 1 generally shows an anterior plan view of a patient's chest showing insertion of the apparatus having first and second bodies where one body being inserted via a sub-xyphoid approach and another body being inserted through a femoral or subclavian vein.

FIG. 2 generally shows a side elevation view of apparatus of FIG. 1.

FIG. 3 shows an enlarged, longitudinal cross-sectional side view of the distal ends of the first and second bodies disposed on opposed sides of a layer of tissue associated with the heart in accordance with a first embodiment.

FIG. 4 is an enlarged, cross-sectional view of the distal ends of the first and second bodies along line 4-4 shown in FIG. 3.

FIG. 5 is a schematic view of the magnetic forces occurring at the distal ends of the first and second bodies shown in FIG. 3.

FIG. 6 is an enlarged, transverse cross-sectional view, similar to FIG. 4, showing transverse cross-sectional view of the distal ends of a second embodiment.

FIG. 7 is an enlarged, side view showing the distal ends of a third embodiment.

FIG. 8 is an enlarged, transverse, cross-sectional view along line 8-8 in FIG. 7.

FIG. 9 is an enlarged, posterior view of a pair of pulmonary veins and the adjacent atrial wall with the distal ends of the first and second bodies on either side thereof.

FIG. 10 is an enlarged, cross-sectional view along line 10-10 of FIG. 9.

FIG. 11 is an enlarged, longitudinal cross-sectional side view showing the distal end of a second body of a fourth embodiment of the apparatus.

FIG. 12 is a cross-sectional view along line 12-12 of FIG. 11, showing the distal ends of both first and second bodies of the fourth embodiment.

FIG. 13 is an enlarged, longitudinal cross-sectional side view, showing the distal end of a second body of a fifth embodiment of the apparatus.

FIG. 14 is a cross-sectional view along line 14-14 of FIG. 13, showing the distal ends of both first and second bodies of the fifth embodiment.

FIG. 15 is a posterior view of the distal ends of a pair of pre-shaped bodies which are positioned adjacent the epicardium around a pair of pulmonary veins and adjacent an atrial wall in accordance with a sixth embodiment of the apparatus.

FIG. 16 is an enlarged, longitudinal cross-sectional view along line 16-16 in FIG. 15, further showing the distal end of a flexible body positioned on the other side of a layer of tissue in alignment with one of the pre-shaped bodies.

FIG. 17 is an enlarged, longitudinal cross-sectional side view, similar to FIG. 3, showing distal ends in accordance with a seventh embodiment of the apparatus.

FIG. 18 is a cross-sectional view along line 18-18 in FIG. 17.

FIG. 19 is an enlarged, longitudinal view of the distal end of a first body according to an eighth embodiment of the apparatus.

FIG. 20 is a cross-sectional view along line 20-20 of FIG. 19, further showing a distal end of a second body.

FIG. 21 is a cross-sectional view along line 21-21 of FIG. 19, further showing a distal end of a second body.

FIG. 22 is a schematic view of the magnetic forces occurring at the distal ends of first and second bodies, which are similar to the embodiment shown in FIGS. 19-20, except that a third body is shown having an identical configuration to the second body is also included.

FIG. 23 is a lateral cross-sectional view of the distal ends, similar to FIG. 4, showing a ninth embodiment of the apparatus.

FIG. 24 is a schematic view of the magnetic forces occurring at the distal ends of the apparatus in FIG. 23.

FIG. 25 is an anterior plan view of a patient's chest generally showing insertion first and second bodies according to the modified apparatus shown in FIGS. 26-28, one body using the intercostal approach and the other body being inserted into the femoral or subclavian vein.

FIG. 26 is an enlarged posterior plan view showing the bodies engaging an atrial wall in the vicinity of a pair of pulmonary veins.

FIG. 27 is an enlarged view of FIG. 26 showing the distal ends in greater detail.

FIG. 28 is an enlarged view, similar to FIG. 27, schematically showing the lines of magnetic force.

FIG. 29 is an enlarged, longitudinal cross-sectional view, similar to the view in FIG. 16 in accordance with a tenth embodiment employing a first expandible member on a lower surface of a first or epicardially-disposed body, the expandible member being shown in an inflated position.

FIG. 29A is an enlarged, transverse cross-sectional view along line 29A-29A in FIG. 29.

FIG. 30 is an enlarged, longitudinal cross-sectional view of the embodiment in FIG. 29, employing a second expandible member on an upper surface of the first or epicardially-disposed body, the second expandible member being shown in an inflated position and the first expandible member being shown in a deflated position.

FIG. 30A is an enlarged, transverse cross-sectional view along line 30A-30A in FIG. 30.

FIG. 31 is a posterior view of the heart showing a pair of pulmonary veins surrounded by the distal ends of right and left curved, epicardically-disposed bodies in accordance with the embodiment of FIGS. 29-30.

FIGS. 31A and 31B show enlarged views of the tips and heels, respectively, in FIG. 31 and illustrate the lines of magnetic force.

FIGS. 32-33 show the method of ablating the right and left pairs of pulmonary veins employing the embodiment of FIGS. 29-31.

FIG. 34 shows several different ablation lesions to the left atrium, as seen from a posterior view.

FIG. 35 is a posterior view of the heart showing the method for making the lesions of FIG. 34 employing up to six epicardially-disposed bodies in accordance with the embodiment of FIGS. 29-31.

FIG. 36 is an enlarged, longitudinal cross-sectional view of a distal end of a flexible epicardially-disposed body, further including a guide facility receiving channel.

FIG. 37 is a longitudinal cross-sectional view of a further embodiment of the apparatus.

FIGS. 37A-37B are transverse cross-sectional views along lines 37A and 37B in FIG. 37.

FIG. 37C is a partial top view of the embodiment of FIG. 37.

FIGS. 38-40 are partial side views of the embodiment of FIG. 37 with portions of the apparatus shown removed.

FIG. 41 shows a side view of the embodiment including a piercing member.

FIG. 42 is an end view of FIG. 41.

FIGS. 43-47 show the steps of ablating a layer of tissue using the embodiment of FIGS. 37-42.

FIGS. 48-52 show alternate steps of ablating a layer of tissue using the embodiment of FIGS. 37-42.

FIGS. 53 is a longitudinal side view of a modification to the embodiment of FIG. 37-42 which employs an expandible member.

FIG. 53A is a transverse cross-sectional view along line 53A-53A of FIG. 53.

FIG. 54 shows the step of ablating a layer of tissue using the modified embodiment of FIGS. 53A and 53A.

### DETAILED DESCRIPTION OF THE EMBODIMENTS

The present invention provides a method and apparatus for ablating tissue, and in particular tissue associated with the heart. Although the method for ablation will be described by way of example but not limitation in relation to the atrial tissue adjacent one of the right and left pulmonary veins, ablation of other areas of the heart are also possible.

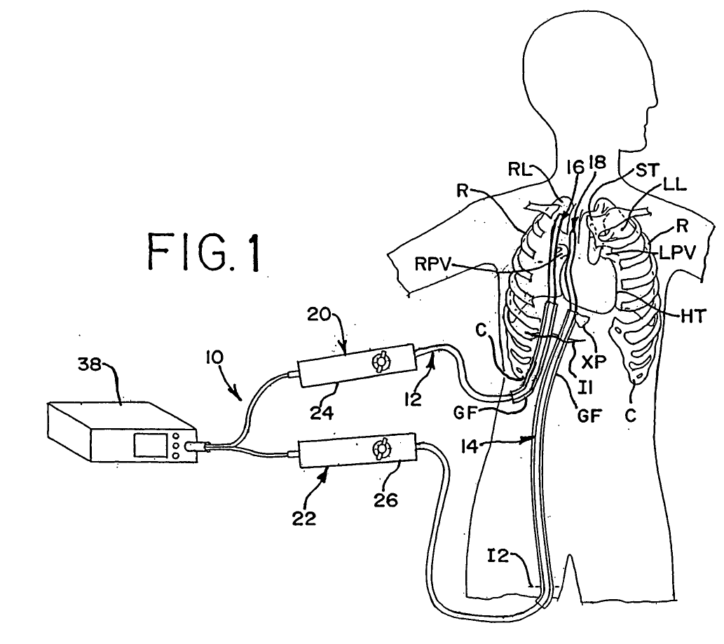

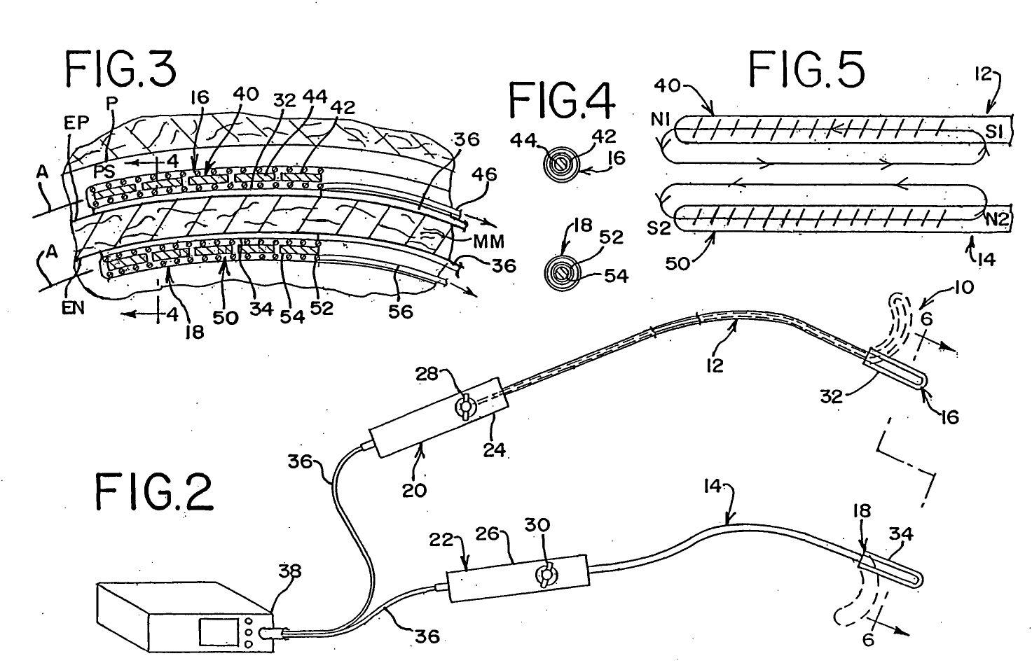

FIGS. 1-2 generally illustrate an ablation apparatus, generally indicated at 10, having first and second elongated bodies, generally indicated at 12 and 14, respectively. Each body has a corresponding distal end 16 and 18 and a proximal end 20 and 22 and is preferably flexible along its length. The proximal end of each body carries a corresponding handle member 24 and 26. The handle member preferably is adapted for moving the distal end by any one or more of various pneumatically, hydraulically or mechanically activated elements (not shown). By way of example and not limitation, movement of the distal end may be aided by a wire, spring mechanism, actuating linkage and or other actuation methods apparent to one skilled in the art. A control knob 28 and 30 is preferably operatively connected to the distal end for articulation of the distal end, as shown in dotted lines in FIG. 2.

Each of the first and second bodies respectively carries an elongated first and second ablation member 32 and 34. Each ablation member is preferably located at the distal end of each body. The ablation member 32 and 34 is preferably an electrode and may be made of a coil, flexible strip of conductive material, or a series of conductive material which are connected to each other. Leads 36 extend from each ablation member 32 and 34 through the respective body to an ablation activation source, generally indicated at 38. The ablation activation source can be provided by any conventional methods such as, but not limited to, a bipolar RF energy generator, a laser source, an ultrasound generator, an electrical voltage source, a microwave generator, and a cryogenic fluid source. For example, the bipolar energy source is energized so current flows through the tissue between the electrodes and ablates the tissue therebetween.

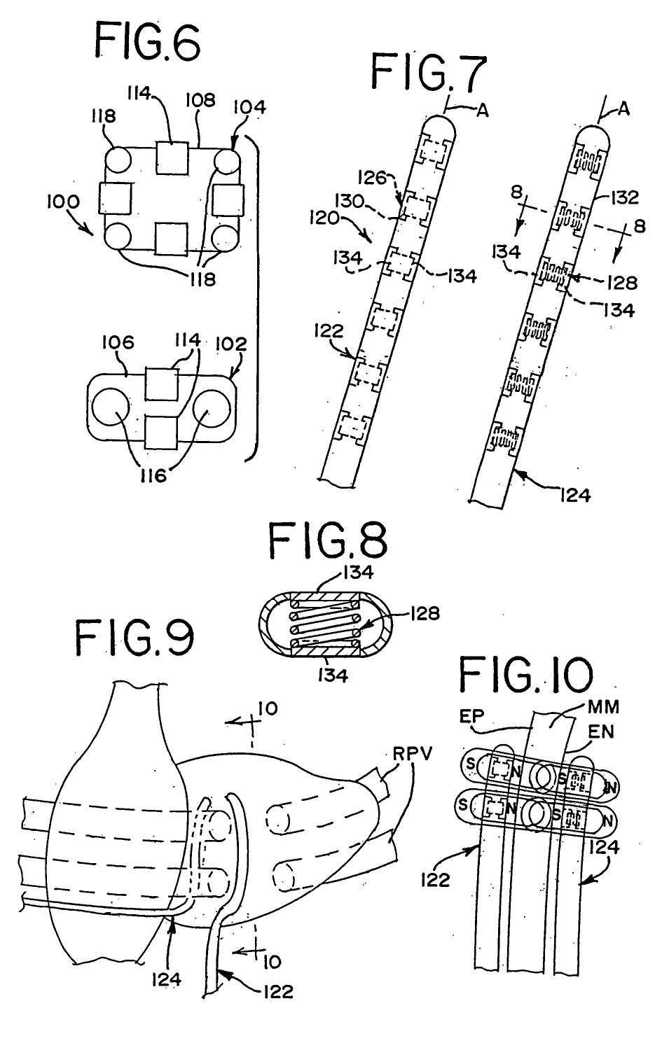

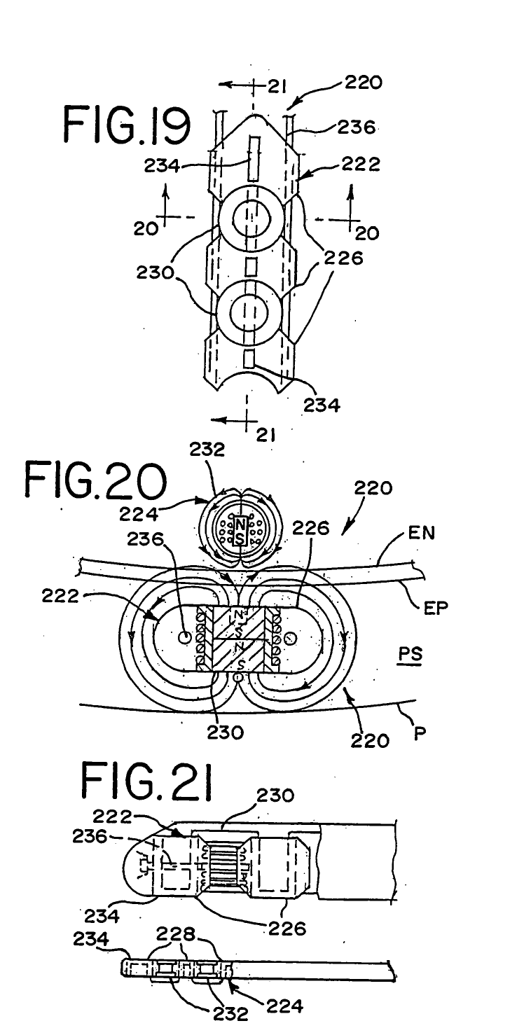

In accordance with a more specific aspect of the invention, FIGS. 3-5 illustrate various views of the distal ends 16 and 18 of a first embodiment of the apparatus. In FIG. 3, first and second bodies 12 and 14 have an exterior surface which defines an interior cavity and each body defines a corresponding axis A along its length. The shape and orientation of the bodies may vary apart from that shown in the views. By way of example and not limitation, the transverse shape of the bodies, which is shown in FIG. 4 as circular, may also be oblong or elliptical, as shown in FIGS. 8 and 20, square or rectangular, as shown in FIG. 6, arcuate, or other geometries, such as the examples shown, not by way of limitation, in FIGS. 12, 14, 23-24 and 26-28 which utilize planar, concave or convex contours, or a combination thereof on the surface or surfaces of the apparatus which contact the layer tissue. Other shapes or geometries are also possible.

In FIG. 3, the distal end 16 of the first body 12 further includes a source of magnetic force, generally indicated at 40. The source of magnetic force 40 includes an insulated coil wire 42 which is oriented longitudinally with the axis A of the body and is wrapped around an iron core 44, which is made of at least one iron cylinder, and preferably, made of a series of iron cylinders oriented longitudinally along the axis A. Electrical conductors 46 extend from a proximal end of the wire 42 through the body and electrically connect to the positive and negative sides of a current source, preferably a direct current source, (not shown) which is supplied by any conventional methods, such as a generator, battery or the like and is positioned outside the patient's chest in a manner similar to the ablation activation source 38 (FIGS. 1-2).

Although not shown in FIG. 3, it is understood that the lines of magnetic force, as shown in FIG. 5, will be generated upon activation by the current source. The flow of current through the wire creates a temporary magnet, conventionally known as an electromagnet. In FIG. 5 the magnetic force lines which are generated include lines originating from a north pole N1 and directed toward a south pole S1, as shown in FIG. 5. The electromagnet may be referred to as a temporary magnet because the magnetic force created by the electromagnet is maintained as long as the current source is applied. When the magnetic force is no longer required, it can be discontinued upon disconnection of the current source. Alternatively, as will be described below, the source may be configured as a permanent magnet which maintains a magnetic force without disconnection.

In FIGS. 3-5, the distal end 18 of the second body 14 carries a magnetically attractive element, generally indicated at 50, which is responsive to the magnetic force of the source 40. In this embodiment, the magnetically attractive element 50 is in the form of an electromagnet which is similar to the source of magnetic force 40 except that this element has a reversed polarity relative to the polarity of the source. The magnetically attractive element 50 has a corresponding insulated coil wire 52, iron core 54, and electrical conductors 56 extending from the coil wire to positive and negative sides of the current source, which may be the same current source used for the first body. The coil wire 52 and iron core 54 are generally disposed in coaxial relation with each other in alignment with the axis A of the second body.

Although FIGS. 3-5 show both the source of magnetic force 40 and the magnetically attractive element 50 as temporary magnets, other variations in the types of magnets also are contemplated and will be described in further detail below with other aspects of the invention. By way of example and not limitation, it is noted that each of the source and the magnetically attractive element may be either a temporary magnet or a permanent magnet, or a combination of the two, which are magnetically attracted to each other. Alternatively, it is also possible that either of the source or the magnetically attractive element is made of a magnetic material. By magnetic material it is meant that the material is affected by or responsive to a magnet. Thus, it is possible that one body carries magnetic material which is attracted by a type of magnet which is carried by the other body. In any event, the source of magnetic force 40 and the magnetically attractive element 50 are adapted for magnetic attraction to each other.

In FIG. 3, the relative positions of the source and magnetically attractive element are generally at the distal end of the first and second bodies, respectively. The magnetic attraction causes the first and second bodies to be drawn closer together in the vicinity of their distal ends and align the electrodes on either side of the tissue layer to be ablated.

During positioning of the bodies, the distal ends of the bodies will repel each other when like poles are in the vicinity of each other. Conversely, the magnetic attraction causes the unlike poles of the magnets to align thereby aligning the distal ends of the bodies relative to the magnetic poles. The magnetic force allows for clamping the tissue to be ablated between the distal ends of the first and second bodies. While a magnetic attraction between the distal ends is preferred, it is not limited thereto. Either of the source 40 or the element 50 may be located along its respective body at any location to allow for clamping at other locations between the first and second bodies, and such other locations are preferably but not exclusively in alignment with a respective electrode. Moreover, it is contemplated that more than one source 40 and/or more than one magnetically attractive element 50 may be located on the respective body and these may establish one or more magnetic attractions therebetween.

As shown in FIG. 5, the magnetic attraction between the temporary magnets is based on the simple principles of magnetism whereby like poles repel each other and unlike poles attract one another. The repulsion or attraction depends on the strength of the magnetic force and the distance between the bodies. The magnetic force of each electromagnet is the product of the number of turns of the coil wire and the amount of current in amperes flowing through the coil wire. Increasing either the number of turns or the current will increase the magnetic force strength. The strength of the magnetic force varies inversely with the separation distance between the source and the magnetically attractive element. As the unlike poles are moved closer to one another, the magnetic force will increase thus increasing the attraction between the poles.

FIG. 5 diagrammatically shows the magnetic attraction between the source 40 and the element 50 of FIG. 3. At the distal-most end of first body 12, a north pole N1 attracts, and is attracted by, a south pole S2 of the distal-most end at the second body 14. At a more proximal position on each body, a south pole S1 of the first body likewise attracts and is attracted by a north pole N2 of the second body.

FIG. 1 illustrates a method of ablating tissue using the first and second bodies of the type shown in FIGS. 3-5. FIG. 1 illustrates a patient's chest including a rib cage R, sternum ST, xyphoid XP, coastal cartilage C, right lung RL, left lung LL and heart HT. One or more incisions are made to allow introduction of the first and second bodies. An incision is made into the patient. In FIG. 1, a first incision I1 is made in the vicinity of the xyphoid or xyphoid process XP and a second incision I2 is made in the upper right leg of the patient. The second incision is made so as to approach the heart intravascularly and, by way of example and not limitation can be accomplished by inserting one of the bodies into a femoral or subclavian vein to the right atrium and by piercing a thin opening through the septum, generally known as the fossa ovalis to access the left atrium. Other approaches may be used utilizing conventional techniques known to those skilled in the art and will depend on which areas of the heart are selected for ablation. By way of example and not limitation, access to the left atrium FIG. 1 shows examples of incision locations and are not intended as a limitations as other approaches and incision locations may be utilized such as, for example, intercostal, intravenous and other minimally invasive approaches as well as more invasive approaches such as open chest procedures or approaches which remove all or a portion of the rib cage. The approach(es) used may depend on other factors involved in the surgical procedure such as which area(s) require ablation and the directional approach which is used to reach the ablation site. The incision may be performed by one of several medical instruments such as a scalpel or the like. Once the incisions are made, each opening defines an instrument receiving passage which the first and/or second bodies can be inserted for access to the heart HT for ablation.

In FIG. 3, one example is shown which positions the first and second bodies on either side of a heart wall, and preferably, on each of the epicardial and endocardial surfaces on opposite sides of the myocardium although numerous other ablation sites are also possible. The first body 12 is positioned within the intrapericardial space PS adjacent the epicardium by a suitable incision into the pericardium, and the second body 14 is positioned adjacent the endocardium EN, preferably within the left atrium, for ablating of a portion of the heart wall or myocardium MM therebetween. The source of magnetic force 40, here in the form of an electromagnet, is positioned on one side of the layer of tissue. The magnetically attractive element 50, which also happens to be an electromagnet in this embodiment, is positioned on the other side of the layer of tissue and is responsive to the source. The magnetic attraction between the two electromagnets is activated by the current source which creates an electromagnet at the distal end of each body, as previously described.

The magnetic attraction between the distal ends also supplies clamping pressure to the tissue layer so as to effectively clamp the tissue layer between the distal ends. The clamping pressure may be regulated by the strength of the electromagnetic field between the bodies. It may be desired to move the first and second bodies into proximity with the selected ablation site prior to activating the electromagnetic force. Then the electrodes can be activated for ablation by the ablation activation device. The tissue layer is ablated by the electrical connection between the electrodes disposed on opposite sides of the tissue layer.

The ablation activation device preferably is adapted for sensing the conductance by the electrodes using a lower ablation energy level prior to full activation of the electrodes. In this way, the device senses whether the electrodes of each body are in sufficient electrical connection with each other across the tissue layer, and thus, sufficiently aligned relative to the cardiac tissue selected for ablation. A predetermined minimum conductivity may be selected below which ablation will not occur. The conductivity is measured and may be compared to the predetermined minimum conductance. The strongest conductance will be achieved when the electrodes are aligned relative to each other. If the electrodes are not in electrical connection or the conductivity is too low, then the device registers that the circuit is open and/or is reflecting too much electrical energy and ablation will not occur. The bodies should be repositioned which can be performed by pulling the magnets apart. Although not required, the current source generating the electromagnet may be disconnected or turned off during repositioning. The ablation activation device will continue to monitor the electrical conductance between the electrodes until the desired conductance is achieved. Thus, if the electrical connection is weak or non-existent, then the device permits repositioning of the bodies prior to ablation.

The first and second bodies may also provide feedback that permits the user to gauge the completeness (i.e., degree of transmurality of the ablation) of the ablation lesion. The feedback generally results from the non-conductive scar tissue which blocks electrical signals and causes the impedance of the tissue to increase. The increase in impedance is reflected by the drop in current. The impedance can be measured, calculated and stored, simultaneously during the creation of the ablation lesion, so as to determine when ablation is complete and transmural. See e.g., U.S. patent application Ser. No. 10/038,506, filed on Nov. 9, 2001, to the same inventor as herein listed above, and U.S. Pat. No. 5,403,312, which are incorporated by reference herein.

Other ablation sites are also possible. Ablation may be performed at one or more tissue locations to create a plurality of ablation lines such as, for example, for treating atrial fibrillation. These ablation lines may be disposed to create an electrical maze in the atria such as that utilized in the Maze procedure. Although the present invention is generally shown as ablating the left atrium LA adjacent the left pulmonary veins LPV, it is realized that the method of ablation may be performed on other areas of the heart. These areas include but are not limited to the atrium adjacent the right pulmonary veins, the left atrial appendage, the right atrial appendage, and other heart locations. In addition, various viewing instruments may be inserted into the intrapericardial space for visual monitoring of the selected ablation site, before, during and/or after ablation.

In FIG. 1, the method further may employ at least one guide facility, generally indicated at GF, so as to further assist in guiding the first and second bodies to the selected tissue. Each body may be assisted by its own guide facility or by the same guide facility. By way of example and not limitation FIG. 1 shows a cylindrical guide facility being advanced to the selected cardiac location from each incision. Then the first and second bodies are inserted into each respective guide facility GF and follow the path created by that guide facility. Alternately, the guide facility may be received within a channel or lumen formed within at least one of the first and second bodies and will be described further below. The guide facility is generally made of a flexible material to facilitate positioning of the guide facility into the patient. Several types of guide facilities are possible including but not limited to a wire, a tube, surgical tape, or the like. The guide facility further may include an endoscope, light source, grasper or other instruments which facilitate locating of the bodies. The guide facility may be disengaged or withdrawn from either one or both of the instrument receiving passages prior or subsequent to positioning of either the first or second bodies. It is also possible that the guide facility is received within a channel defined within either of the bodies, a suitable insertion sleeve, trocar, or like device.

Turning to FIG. 6, a second embodiment of the apparatus is shown generally at 100. The first and second bodies, respectively indicated at 102 and 104, which are similar to FIGS. 1 and 2 except that the respective distal ends 106 and 108 have different configurations relative to each other. The second embodiment generally shows distal ends having two or more ablation members, sources of magnetic force and magnetically attractive elements which are carried at different locations. Various other configurations may be possible so as to facilitate engaging the distal ends into different orientations and configurations depending on the site which is selected for ablation.

In FIG. 6 the distal end 106 of the first body 102 includes two ablation members 114 carried along the circumference of the distal end, in a manner similar to FIG. 3, except that one electrode is disposed at each of an upper and lower surface of the body. Each ablation member 114 is generally parallel to the axis of the body. The distal end 106 further includes two sources of magnetic force 116 in the form of electromagnets which are spaced on either side of the two ablation members and oriented parallel to the axis of the body. In FIG. 6, the distal end 108 of the second body 104 has four ablation members 114 which are spaced around the circumference of the distal end. Magnetically attractive elements 118 are alternated between the ablation members 114 on the distal end and oriented parallel to the axis of the body.

In FIG. 6, the magnetically attractive element 118 is made of magnetic material, preferably a composite of iron and stainless steel which is attracted by the electromagnet of the source 116, although other types of magnetic material are also possible and will be apparent to those skilled in the art. The magnetic material may be made from various techniques which are known in the art including and not limited to molding, plating, coating, depositing, or the like. One of the bodies is advanced to one side of the selected tissue and the other body is advanced to the other side of the tissue. The current source is activated and creates an electromagnet at the distal end 106 of the first body 102. The magnetic material in the distal end 108 of the second body 104 is attracted by the magnetic force created by the electromagnet of the first body. The tissue between the distal ends 106 and 108 is clamped as the magnetic force pulls the distal ends of the bodies together. The ablation activation source determines that the electrodes 114 are aligned as previously described by impedance measurement and the tissue is ablated.

FIGS. 7 and 8 show a third embodiment of the apparatus 120. Each of the first and second bodies, respectively indicated at 122 and 124, have sources of magnetic force and magnetically attractive elements, generally indicated by respective reference numbers 126 and 128 and, in this embodiment, are each comprised of electromagnets which have lines of magnetic force, as shown in FIG. 10, upon activation by the current source.

The embodiment of FIGS. 7 and 8 is similar to the first embodiment of FIGS. 3-5 except that the coil wires of each electromagnet are disposed along an axis which is transverse to the axis A of each distal end, respectively indicated at 130 and 132. The electromagnets in each distal end 130 and 132 are spaced relative to each other along the body axis. Ablation members 134 are also spaced from one another along each distal end and are generally, but not exclusively, aligned with respective electromagnets. The ablation members 134 are positioned in the upper and lower surfaces of the distal end of each body, similar to the distal end 106 described in FIG. 6.

In FIG. 7 the distal end of each body is highly flexible to allow the distal end to conform to the surface of the heart that is selected for ablation and may further be capable of articulation. Movement of each distal end is provided by a suitable linkage (not shown) which preferably extends to a corresponding handle member (FIG. 1-2) which allows for flexible movement of the distal end. The movement may be controlled by the control knob, such as shown in embodiment of FIG. 1-2. Alternatively, the distal end may flexibly moved by hand positioning by the surgeon into a desired shape and be adapted to retain that shape until repositioned.

FIGS. 9-10 show another example of a method of ablation using the type of apparatus shown in FIG. 7. The method may employ any of the previously described approaches in any combination. Whichever approach(es) are used, the first and second bodies 122 and 124 are advanced to the atrium adjacent the right pulmonary veins RPV. The coil wires in each body are energized by the current source and a magnetic attraction is created between the unlike poles of the first and second bodies 122 and 124, as shown by the lines of magnetic force in FIG. 10. The magnetic attraction increases as the bodies are drawn closer together so as to clamp the region of the atrium AT for ablation. In FIGS. 9-10, the selected ablation location is shown as the region of the left atrium associated with the right pulmonary veins RPV for ablation.

Turning now to FIGS. 11-12, a fourth embodiment of the apparatus generally indicated at 140 is shown having first and second bodies, respectively 142 and 144 (FIG. 12) with corresponding distal ends 146 and 148. A respective source of magnetic force 150 and a magnetically attractive element 152 are each in the form of at least one electromagnet. Each electromagnet is disposed in a yet further orientation than those previously described above. Each of the distal ends of the first and second bodies are identical to one another and, as such, only one will be described.

At least at the distal end 148 of the second body 144 the body has a transverse shape which includes a planar surface facing the tissue layer and a convex surface facing away from the tissue layer. Relative to the axial direction of the body shown in FIG. 11, the electromagnets 152 are oriented in several spaced rows carried by the distal end. As shown in FIG. 12, each row has a coil wire which is curved approximately 180 degrees between its ends and its ends are oriented toward the planar surface. Each row may also be referred to as having a half-toroidal geometry. This shape is not intended to be a limitation as other shapes are also possible. An ablation member 154 is disposed between the ends of the coil wire and is connected to ablation activation source.

In FIGS. 11-12, each body has numerous rows of coil wires. Each row of electromagnets forms lines of magnetic force from the north pole N to the south pole S which lines are schematically indicated in FIG. 12. Relative to the first body, the second body has reversed polarity so as to be magnetically attracted to the first body. It follows logically that the magnetic forces of all the rows aggregate together to form an even larger electromagnet so that the electromagnets of one body are attracted to the electromagnets of the other body. The apparatus 140 may be used in accordance with any of the methods previously described. By way of example, the distal end of the first body 142 is shown within the intrapericardial space PS at one side of the atrial wall and the distal end of the second body 144 is shown inserted into the chamber of the left atrium. The magnetic attraction will provide a clamping pressure to the atrial wall. Thereafter ablation can be performed.

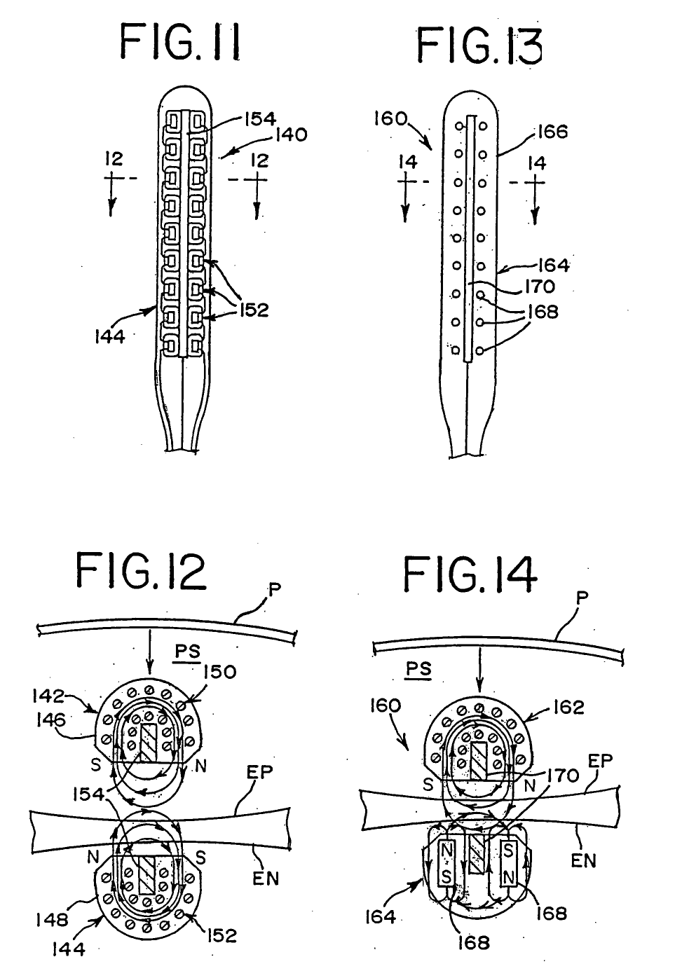

Turning to the fifth embodiment in FIGS. 13 and 14 of a yet further apparatus generally indicated at 160. A first body 162 is similar to the first body described in FIGS. 11 and 12 and need not be described further. A second body 164 includes a distal end 166 having a plurality of magnetically attractive elements 168 in the form of permanent magnets. As shown in FIGS. 13 and 14, the permanent magnets are disposed in an array of columns and rows along the longitudinal length of the distal end 166 of the second body 164. Each row has two permanent magnets which are generally in alignment with the ends of the coil wire of the first body 162. Each permanent magnet has a generally cylindrical shape and is oriented in a transverse direction relative to the longitudinal axis A of the body. As shown in FIG. 14, one permanent magnet is disposed on either side of an ablation member 170 with one magnet having a reversed polarity so that a north pole N of the left permanent magnet of the second body 164 is aligned with the south pole S of the first body 162. Conversely, the south pole S of the right permanent magnet of the second body is aligned with the north pole N of the first body thus providing a magnetic attraction. The permanent magnets may be made of any suitable material such as, for example, rare earth magnets or the like.

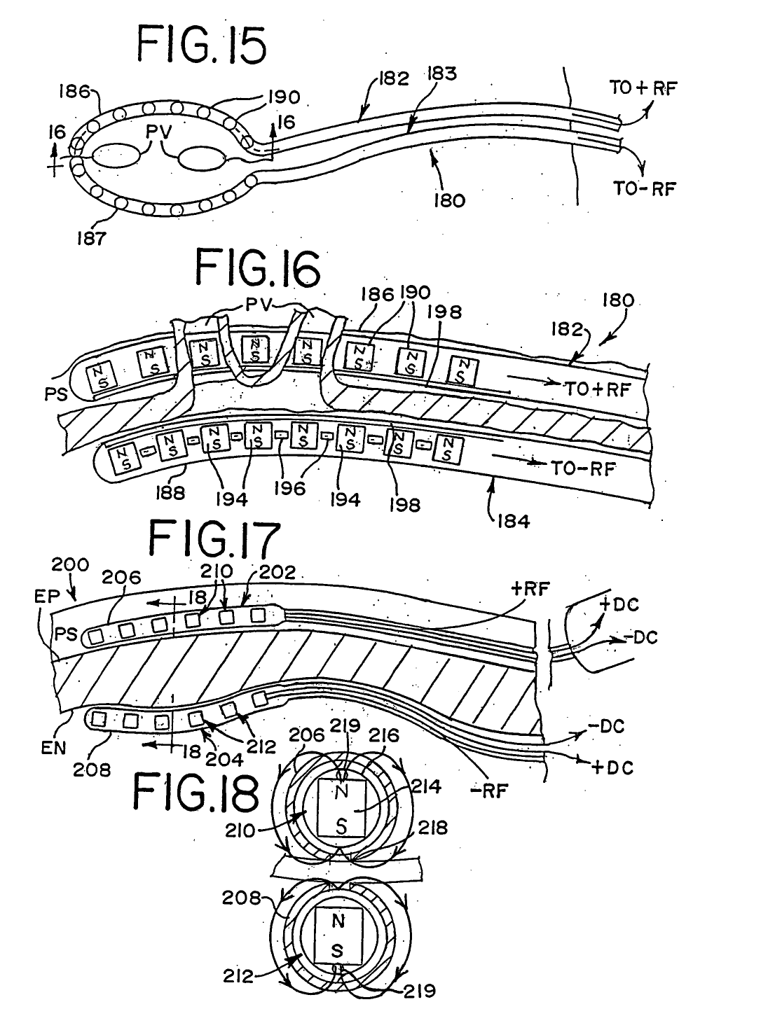

Turning to FIGS. 15 and 16, a sixth embodiment of the apparatus, generally indicated at 180, is shown having right and left distally curved first bodies 182 and 183 and a second body 184 having respective distal ends 186, 187 and 188. The right and left curved first bodies 182 and 183 are preferably, but not exclusively, rigid and flexibly curved first bodies will be discussed below. The right and left curved first bodies are placed around the atria in the vicinity of a pair of pulmonary veins PV. Each curved distal end has a concave surface facing the veins and a convex surface facing away. Other curves are possible and will depend on the selected tissue for ablation.

As shown in FIG. 16, the distal end 186 of the right curved first body 182 has a plurality of sources of magnetic force 190 which may be permanent magnets, temporary magnets or magnetic material which are spaced along the length of the distal end and which have north and south poles oriented transverse to the axis of the body. It is noted that the left curved first body 183 may have a similar orientation and it need not be described further.

The second body 184, as shown in FIG. 16, is preferably flexible along at least a portion of its length and, preferably, at least along the distal end 188. The flexibility of the second body permits its distal end 188 to maneuver into curved alignment with one or both of the curved distal ends 186 and 187 of the first bodies. The curvature of the distal end 188 may be performed or aided by an articulating linkage or by manually positioning.

In FIG. 16 the distal end 188 includes a plurality of spaced magnetically attractive elements 194 which are spaced along the length of the distal end. The magnetically attractive elements are disposed in a transverse direction relative to the axis of the body and may be in the form of permanent magnets, temporary magnets or magnetic material. In FIG. 16, permanent magnets are separately introduced into the internal cavity defined by the second body 184. Although not required, spacers 196 may be inserted between adjacent magnets. A guide facility may be used to slidably advance each magnet and spacer into the second body so as to position the magnets and spacers.

One or both of the rigid bodies 182 and 183 may be inserted via a percutaneous incision within the intrapericardial space PS and engage the atria adjacent a pair of pulmonary veins PV. In FIG. 16, the second body 184 is introduced intravascularly and advanced through a standard trans-septal percutaneous approach to reach the atrial wall in the vicinity of the pulmonary veins although the method may be employed using other approaches as is apparent to one skilled in the art.

As shown in FIG. 16, the magnetic attraction between the north and south poles causes the first and second bodies to clamp the atrial wall. Ablation is performed by ablation members 198 which are disposed on each of the first and second bodies. The magnetic attraction between the rigid, right curved body 182 and the flexible second body 184 can be broken by drawing the bodies apart from one another, or turning off or disconnecting the current source generating a temporary magnet. Alternatively, the magnets within the second body may be removed or withdrawn anteriorly, preferably with the aid of the guide facility to decrease or eliminate the magnetic attraction. In either event, the second body 184 can be disengaged from the right curved body 182 and can be repositioned in alignment with the left curved body 183 with the tissue being clamped therebetween. Then the step of ablation is repeated.

Turning to FIGS. 17 and 18, a seventh embodiment of the apparatus generally indicated at 200 which illustrates a further modification. First and second bodies 202 and 204, respectively, have corresponding distal ends 206 and 208 and a plurality of sources of magnetic force 210 and magnetically attractive elements 212, respectively.

As shown in FIG. 18, the first body 202 has a permanent magnet 214 in the form of a permanent bar magnet having north and south poles N and S. An electromagnet also may be used either in addition to or instead of the permanent magnet and, where it is used, it is realized that electrical connections will be provided to positive and negative sides of a direct current source, +DC and −DC (FIG. 17). A coil wire 216 surrounds the magnet and extends along the distal end 206. At least a portion of the exterior of the body 202 which surrounds the coil is insulated except for an elongated slot 218 which extends along the distal end 206. The slot 218 exposes a portion of the coil thus, creating an electrode for ablation. As shown in FIG. 18, the distal end of the second body has a construction which is a mirror image of the first body. The first and second bodies are connected to a bipolar energy source in order to provide ablation energy.

In addition, each of the first and second bodies 202 and 204 has an elongated guide facility channel 219 which extends along at least a portion of the length of the body. As mentioned earlier, a guide facility may be used with one or both of the bodies so as to facilitate the advance of each body to the selected ablation site. The guide facility will conventionally having two ends and an intermediate portion extending therebetween. One end of the guide facility may be inserted into the guide facility channel of each body or a separate guide facility may be used for each body. As shown in FIG. 17, the first body is inserted into a intrapericardial space PS, and the second body is inserted into the left atrium for ablation of a heart wall or other like location.

In a further modification of the invention, FIGS. 19-20 show a coil wire around one or more permanent magnets to affect the magnetic attraction between the bodies although these embodiments are not exclusively limited to this type of magnetic attraction. Any one or more of the permanent magnets in any of the embodiments described herein may also be modified by the introduction of a coil wire. The coil wire will either increase or decrease the existing magnetic force and will depend on the direction of current which flows through the wire and the direction of force line produced relative to the force lines of the existing magnetic force.

Turning to FIGS. 19-20, an eighth embodiment of the apparatus generally indicated at 220, is illustrated having a first and second body, respectively indicated at 222 and 224. The distal end of each body comprises a plurality of segments, respectively at 226 and 228, alternated in between by an articulation joint, respectively 230 and 232. The segments and joints are disposed at least along the distal end of each body to a more proximal location and permit flexible movement of the distal ends.

Each segment 226 and 228 carries a suitable ablation member 234 such as an electrode connected by leads to an ablation activation device such as a RF generator. Each segment 226 of the first body further has longitudinally extending openings for receiving an articulating linkage 236 such as a cable or the like which extends from the distal end of the first and second body to preferably a handle member (not shown) at the proximal end of the body. The segments 228 of the second body may include openings for an articulating linkage if desired, or alternatively, the second body may be moved by magnetic attraction to the first body or by manual positioning. Each articulating joint 230 and 232 preferably has a circular or cylindrical shape so as to permit articulation of the distal end by way of pivoting movement of the segments relative to each other when the linkages are pulled or pushed either by the articulating linkage, by magnetic attraction or by manual positioning.

In accordance with the previously discussed aspects of the invention, each body carries either a source of magnetic force or a magnetically attractive element, which in FIGS. 20-21 are carried by the articulating joints 230 and 232. As shown by way of example, the articulating joint 230 of the first body 222 has at least one permanent magnet and a coil wire which surrounds the permanent magnet and connects to the positive and negative sides of an electrical current source. The articulating joint 232 of the second body 224 is shown having a magnet with a similar configuration. FIG. 20 shows the lines of magnetic force between the bodies.

The coil wire may increase, decrease or completely cancel out the resulting magnetic force from the permanent magnets depending on the direction of current supplied to the coil wire from the current source through suitable electrical conductors. For example, the coil wire may increase the magnetic force during positioning of the bodies on either side of the tissue layers to be ablated. Conversely, the electrical current source can reverse the current flowing through the coil wires so as to decrease, reverse or completely dissipate the magnet attraction and allow removal of the bodies from the ablation site.

FIGS. 20 and 21 show first and second bodies, respectively 222 and 224, with the first body 222 being disposed adjacent the epicardial surface and the second body 224 being disposed adjacent the endocardial surface. The first or epicardially-disposed body 222 is shown as having a larger diameter than the diameter of the second or endocardially-disposed body 224. The pericardium may be separated from the epicardial surface so as to create an intrapericardial space PS which has sufficient area to accommodate an epicardially-disposed body having a larger diameter. The second body 224 which is positioned within the heart preferably has a smaller size, for example, to permit insertion into a femoral or subclavian vein for travel to the heart. Although other sizes are also possible, the diameter for the first body 222 is preferably approximately 5-10 mm, and the size of the second body 224 is preferably approximately 3 mm.

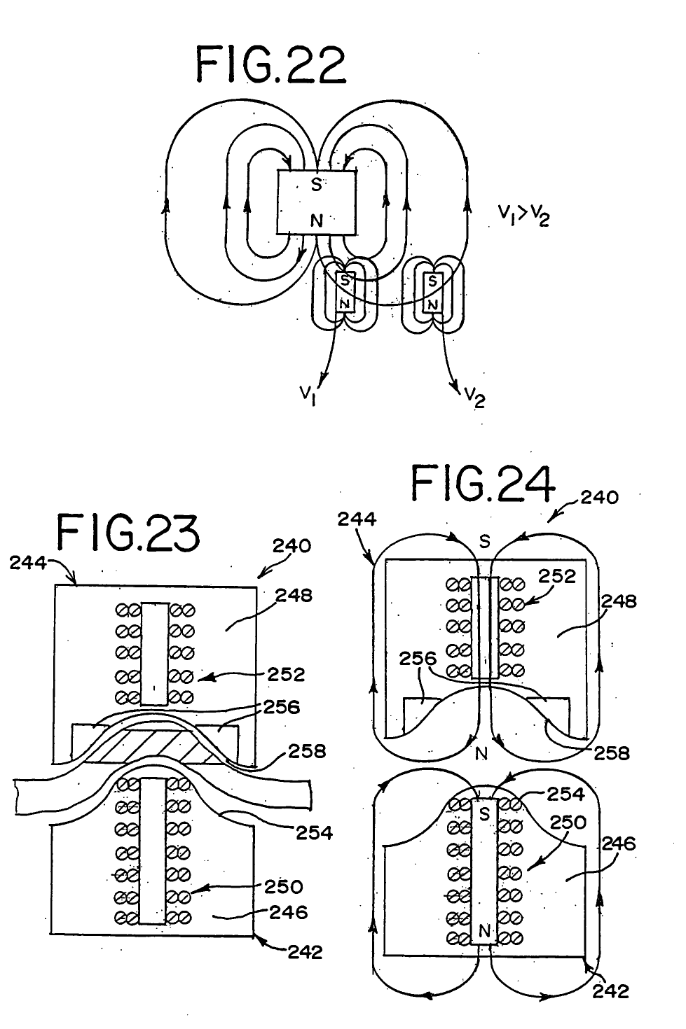

As shown in FIG. 22, the magnetic attraction is schematically shown between a first or epicardially-disposed body of relatively large size and a second or endocardially-disposed body of smaller size. The alignment between the bodies can be facilitated by a sensor or other logic device in operative connection with the magnets or electromagnets. It is contemplated that not all magnets or electromagnets may align due to differences between the bodies, e.g., where the relative separation distance between adjacent magnets varies for each body. The logic device can measure, calculate and store the magnetic field density occurring between the bodies and determine where the magnetic field density is greatest. The magnets or electromagnets of the smaller body moving through the magnetic force lines of the larger body may create a feedback signal which can also be monitored. The device can determine the greatest field density which corresponds to aligned magnets or electromagnets and then energize only the ablation members that are within the greatest field density.

Turning to FIGS. 23 and 24, a ninth embodiment of the apparatus generally indicated at 240 shows first and second bodies, respectively indicated at 242 and 244, and at least a distal end 246 of one of the bodies has a contoured surface and the distal end 248 of the other body has a complementary contoured surface. The first and second bodies respectively include a source of magnetic force 250 and a magnetically attractive element 252. The first body 242 defines a convex surface 254 in the transverse direction of the body. The second body 244 defines a concave surface 258 which is complementary to and generally matches the convex surface 254. As shown in FIG. 23, ablation members 256, preferably electrodes, extend longitudinally at either side of the concave surface. Figure also shows a tissue layer which is clamped between the concave and convex surfaces by the magnetic force, shown by the force lines in FIG. 24. The atrial wall is biased between the electrodes disposed on the concave surface 256 and the tissue layer is ablated.

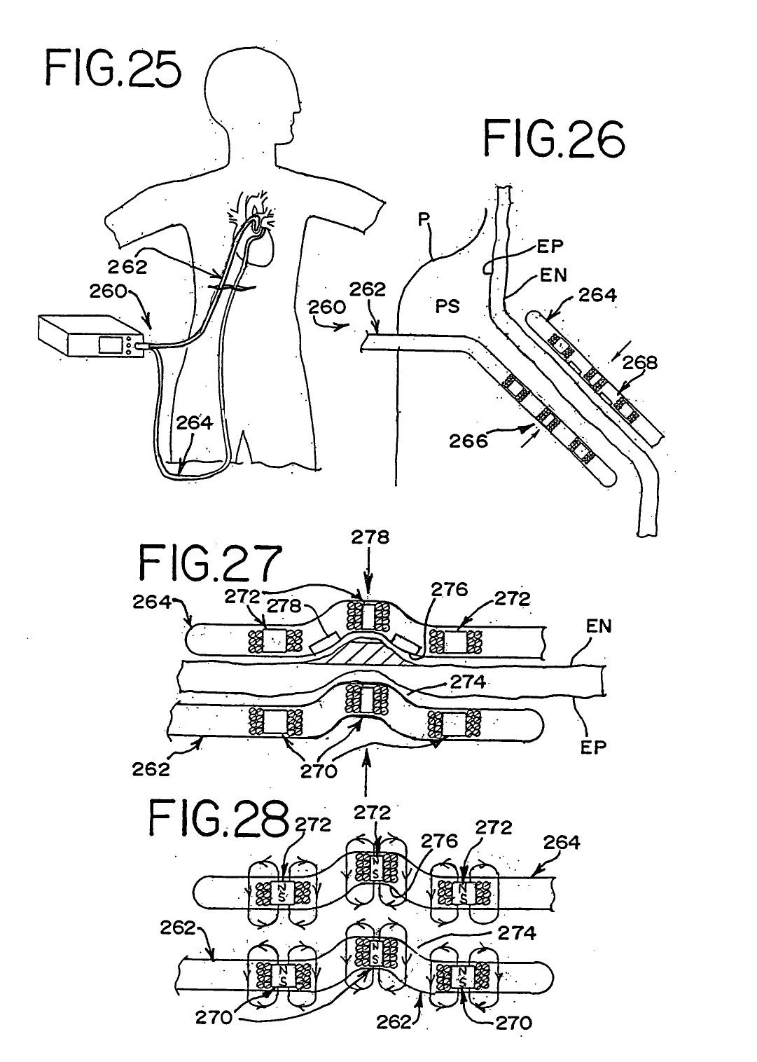

FIGS. 25-28 illustrate an apparatus 260 which is similar to the apparatus in FIGS. 23 and 24 except that the first and second bodies, respectively 262 and 264, each respective distal end 266 and 268 has a plurality of sources of magnetic force 270 and magnetically attractive elements 272 which are spaced relative to each other on the respective distal ends, although other orientations and configurations are also possible and will be apparent to one skilled in the art. Similar to the embodiment shown in FIGS. 23 and 24, the first body 262 has a convex surface 274 and the second body 264 has a concave surface 276 which complements the convex surface and compresses the atrial wall tissue into the convex surface so that the tissue for ablation is disposed between electrodes 278 disposed on the concave surface. FIG. 28 illustrates the lines of magnetic force created between the sources of magnetic force and the magnetically attractive elements for alignment of the surfaces 274 and 276 on opposite sides of the tissue layer.

FIGS. 25 and 26 illustrates introduction of the first and second bodies 262 and 264 into the patient's chest which employ a sub-xyphoid approach and femoral or subclavian vein approach, respectively. The first body 262 which is introduced via the sub-xyphoid approach preferably has a rigid profile although it may also be flexible in accordance with other embodiments discussed herein. The second body 264 which is introduced via the femoral or subclavian vein preferably is flexible by way of an articulation linkage extending through the body, manual manipulation of the distal end, and/or maneuverable by way of its magnetic attraction to the rigid first body, as previously discussed relative to earlier embodiments. FIG. 25 show the first and second bodies engaging the left atrium in the vicinity of the left pulmonary veins although it is intended that the bodies may engage other areas of the patient.

FIGS. 25-28 also show overlapping of the distal ends of the first and second bodies from directionally-opposite approaches. Nonetheless, the sources 270 and elements 272 are aligned by way of their complementary surfaces and magnetic attraction as described above. For example, the distal-most source 270 of the first body 262 aligns with a more proximal element 272 of the second body 264 and vice versa. The intermediate source 270 and element 272 also align. The flexible second body conforms to a complementary shape and ablation of the tissue layer is performed.

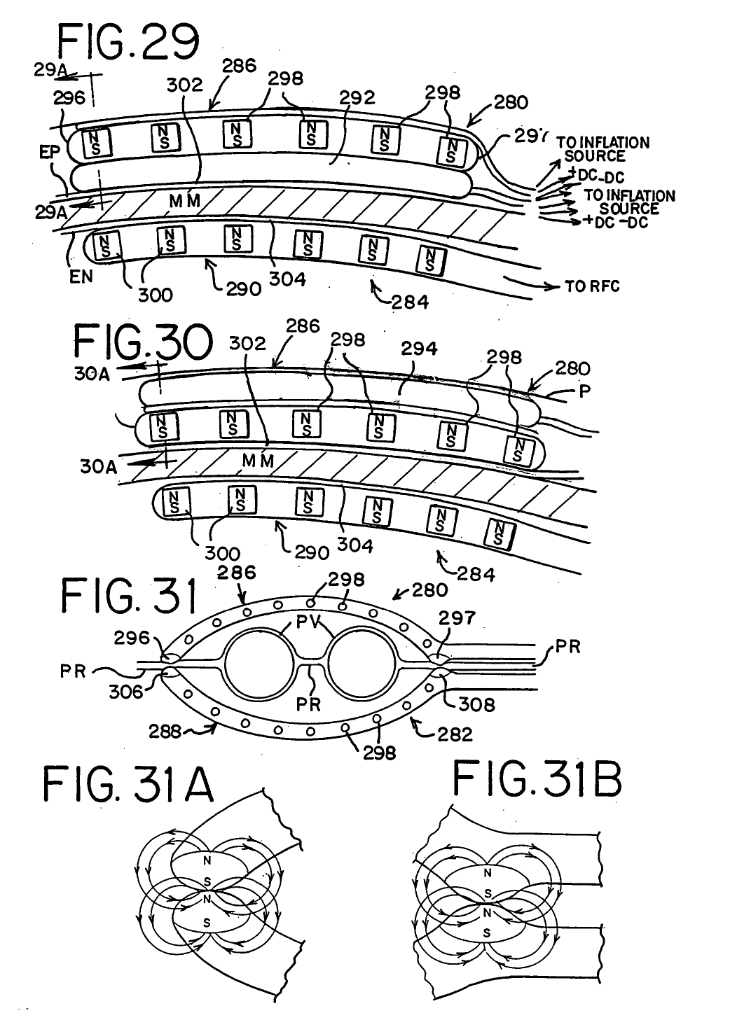

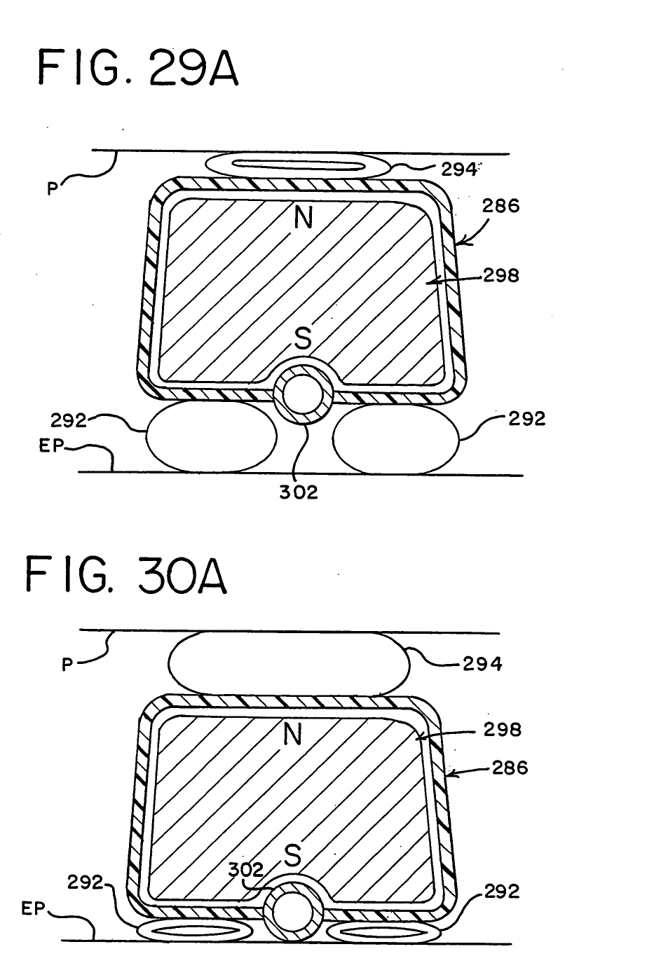

FIGS. 29-31 show another method and apparatus in accordance with a tenth embodiment of the present invention. The embodiment of FIGS. 29-31 is similar to the embodiment shown in FIGS. 15-16 in that it includes right and left distally curved first bodies, generally indicated at 280 and 282, only one body 280 being shown in FIGS. 29-30 and both bodies 280 and 282 being shown in FIG. 31, and further includes a flexible second body, generally indicated at 284, shown in FIGS. 29-30. Each of the first and second bodies 280, 282 and 284 have respective distal ends 286, 288 and 290, as shown in FIGS. 29-31. The right and left curved bodies facilitate positioning of the bodies on the epicardial surface EP of the heart around a pair of pulmonary veins PV, as shown in FIG. 31, and may be rigid or flexible. Other shapes are also possible in order to reach other surfaces of the heart. Each of the right and left curved bodies are similar to one another except for their respective shapes and, as such, only the right curved first body will be described in detail.

In FIGS. 29-30, the distal end 286 of the right curved first body 280 is located on one side of the myocardium MM and preferably engages the epicardial surface EP of the heart. The distal end 290 of the flexible second body 284 is located on the opposite side of the myocardium MM and preferably engages the endocardial surface EN of the heart. The first body 280 further includes one or more expandible members 292, 294. The expandible member may include an inflation lumen, which fluidly communicates between a balloon and an inflation source, an expandible cage-like member or members, a spring-actuated expandible member or members or the like which is adapted for expanding and retracting as desired.

Each expandible member 292 and 294 preferably extends along the longitudinal axis of the first body 280 and, more preferably, extends along the distal end 286 from a tip 296 of the first body to a more proximal location. For example, each expandible member 292 and 294 preferably extends along the curved distal end to a proximal location or heel 297. The heel 297 preferably, but not exclusively corresponds to the most proximally located source of magnetic force 298.

As shown in FIG. 29, the first expandible member 292 is located on a first or lower surface of the first body 280 which is normally positioned on one side of the tissue layer. In FIG. 30, the second expandible member 294 is located on a second or upper surface of the first body 280 which is generally opposite the first surface and normally positioned away from the epicardial surface such that it faces the pericardium P. Each expandible member 292 and 294 is also located on an opposite side of the plurality of sources of magnetic force 298.

In FIGS. 29A and 30A, the lower expandible member 292 has a bifurcated shape having two elongated branches which extend along either side of the ablation member 302. In FIGS. 29A and 30A the ablation member is shown as an electrode coil which extends through an insulated wall of the first body 280. The branches of the first expandible member 292 preferably join together at a more proximal location of the first body 280. In FIGS. 29A and 30A the second expandible member 294 is shown having an elongated, non-branched shape. These shapes are shown by way of example and not limitation. It is contemplated that other shapes may also be utilized for each expandible member.

Each expandible member 292 and 294 is further connected, depending on the type of expandible member which is employed, by conventional inflation lumen and an inflation source located outside of the patient or by a suitable or actuating linkage or the like. Inflation fluids may include saline, air or the like. In any event, expansion and/or retraction of the expandible member is effected by fluid or actuation, as desired.

In FIGS. 29-30, the elongated flexible second body 284 includes a plurality of magnetically attractive elements 300 which are responsive to the plurality of sources of magnetic force 298, each of which may by provided in accordance with any of the previously described embodiments. In addition, FIGS. 29-30 show the flexible second body 284 as generally having a smaller transverse area than that of the first body 280, which has also been previously described above, although other cross-sectional areas for the second body 284 are also possible and may depend on the specific approach employed to reach the selected ablation site. Each of the first and second bodies further include respective ablation members 302 and 304 which are located on the respective distal ends or tips 286, 290 and may be made of flexible strips of conductive material or exposed sections of coil, as shown and described in the embodiment of FIG. 18.

FIGS. 29-35 show the method for ablating a layer of tissue using the apparatus shown in FIGS. 29-31. The first body 280 is introduced and advanced to an epicardial surface EP of the heart adjacent one side of the tissue which is selected for ablation. As shown in FIG. 29, the first expandible member 292 is an elongated balloon disposed at the lower surface of the first body 280 adjacent the epicardial surface EP. The balloon extends longitudinally along the distal end. Inflation or expansion of the balloon creates a space or cushion between the sources of the magnetic force 298 and the epicardial surface EP. The relative position of the first body 280 within the intrapericardial space PS remains unchanged. The expandible member 292 exerts pressure against the epicardial surface EP and pericardium P to assist in anchoring the first body 280 into position adjacent the epicardial surface EP at the selected ablation site. The ablation member 302 preferably remains in contact with the epicardial surface EP.

After inflation, the second body 284 is preferably advanced to the endocardial surface EN on the other side of the tissue layer selected for ablation. The second body 294 is maneuvered into the desired position so as to generally align its distal end 290 with the distal end 286 of the first body 280 on the opposite side of the tissue. Inflation of the first expandible member 292 minimizes or eliminates the influence of the magnetic attraction during positioning of the second body by increasing the distance through which the magnetic force acts.

In FIG. 29, the increased distance between the sources of magnetic force 298 and the magnetically attractive elements 300 is sufficient to prevent movement due to magnetic attraction between the first and second bodies. The inflation thickness of the expandible member is preferably approximately 1 cm although other thicknesses are also possible and will depend on the strength of the magnetic attraction between the first and second bodies.

In FIG. 30, the first expandible member 292 is deflated. Deflation moves the sources of magnetic force 298 in closer contact with the epicardial surface EP, and re-establishes the magnetic attraction, with the magnetically attractive elements 300 of the second body 284. The magnetic attraction between the first and second bodies 280 and 284 should align the respective distal ends 286 and 290. The endocardially-disposed second body 284 is preferably flexible so as to be adapted to conform to the curved shape of the epicardially-disposed body by its magnetic attraction. The surgeon may feel a tactile sensation from the two bodies being magnetically draw together. If there is no magnetic attraction, then the first expandible member may be re-inflated again to permit repositioning of the second body 284.

During deflation of the first expandible member 292, the second expandible member 294 may be inflated, as shown in FIG. 30. Inflation of the second expandible member 294 during deflation of the first expandible member 292 assists in maintaining a constant pressure on the epicardial surface EP and pericardium P by so as to keep the first body 280 anchored in the desired position.

The lower ablation energy level may be employed prior to full activation of the ablation members to monitor the impedance and conductance, as previously described. Once the first and second bodies 280 and 284 are in the desired position, ablation members are activated to ablate the selected location. When ablation is completed, the first expandible member 292 is preferably re-inflated to the position shown in FIG. 29 to move the sources of magnetic force away from the epicardial surface EP. This allows the magnetic attraction between the first and second bodies 280 and 284 to disengage for repositioning of the second body 284 to another location, e.g., adjacent the left atrium in alignment with the left curved first body 282 of FIG. 31. The above described steps are repeated so that the endocardially-disposed body flexibly conforms to the epicardially-disposed first bodies 282 and creates a continuous ablation lesion on the left atria which encircles the pair of pulmonary veins.

FIG. 31 shows the right and left curved bodies surrounding a pair of pulmonary veins PV so as to assist in creating a virtually continuous ablation lesion at the atrial tissue surrounding the veins. The tip 296 and heel 297 of the right curved first body 280 is preferably magnetically attracted to a corresponding tip 306 and heel 308 of the left curved first body 282. In this regard, any of the previously described magnets and/or magnetic material or combination thereof is carried by each of the tips 296 and 306 and heels 297 and 308. The magnets or magnetic material are oriented so that the respective tips 296 and 306 and heels 297 and 308 are magnetically attracted to one another. In the example shown in FIG. 31, the tips 296 and 306 are magnetically attracted to one another, and so are the heels 297 and 308, with the lines of magnetic force being shown in FIGS. 31A and 31B. The magnetic attraction assists in aligning and/or clamping the respective tips and heels of the bodies 280 and 282 together from opposite sides of the pericardial reflection PR. The operator may feel a tactile sensation or clicking sound as the respective tips and heels of the two bodies are magnetically draw together.

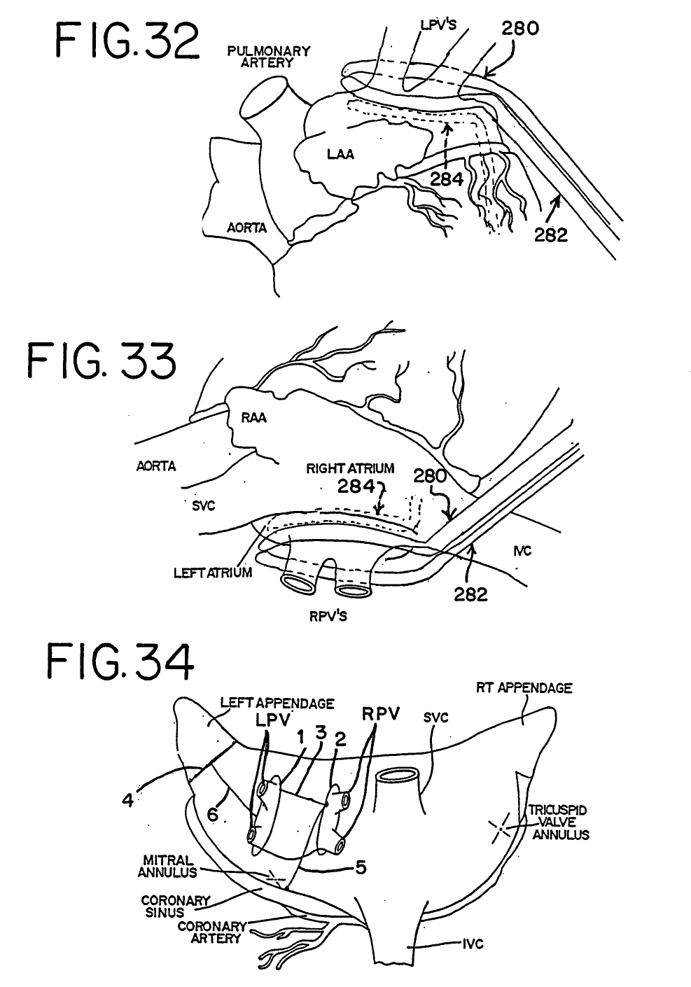

The method described in FIGS. 29-31 may be employed for many different areas of the heart and the epicardially-disposed body may employ different shapes specifically suitable for each area. FIGS. 32-33 show placement of the several epicardially-disposed right and left curved first bodies 280 and 282 around the left and right pairs of pulmonary veins, respectively. The right and left curved first bodies may be rigid or may be flexible with shape retention characteristics. Where the epicardially-disposed bodies is rigid, differently shaped bodies may be employed depending on which areas are being selected for ablation. A flexible body may be manually pre-shaped into a desired shape while outside the patient and have sufficient rigidity to retain the desired shape until manually repositioned. Alternatively, a suitable articulating linkage may be used to control movement of the distal end. It is further possible that a guide facility may be shaped as desired and be adapted to extend through each body so that the body takes the shape of the guide facility, as described below in FIG. 36.

FIG. 34 shows a posterior view of the heart including the superior vena cava SVC and inferior vena cava IVC in addition to other areas of the heart, as shown. A number of ablation lesions can be created according to any one or more of the above described methods and some examples of ablation lesions are shown in FIG. 34 as follows: a lesion 1 encircling the left pulmonary veins; a lesion 2 encircling the right pulmonary veins; a lesion 3 extending between the left and right pulmonary veins; a lesion 4 encircling the left atrial appendage; a lesion 5 extending downward from the pulmonary veins to the mitral annulus; and a lesion 6 extending from the left pair of pulmonary veins LPV to the left atrial appendage. Other ablation sites are also possible and will be apparent to those skilled in the art.

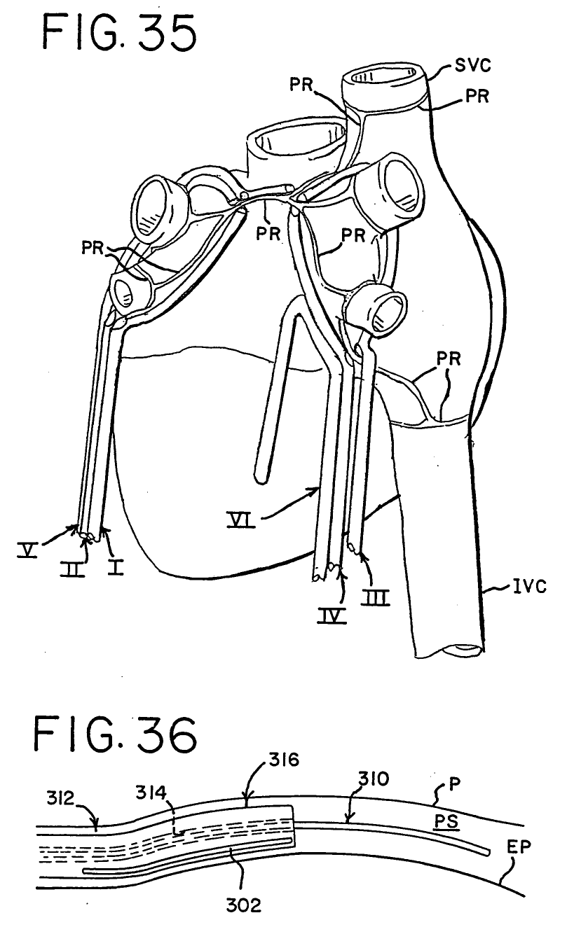

In accordance with a further aspect of the apparatus and method, up to approximately 5 or 6 epicardically-disposed bodies may be introduced into the patient's body to effect several ablation lesions on the surfaces of the heart. Six epicardially-disposed bodies are shown by way of example and not limitation in FIG. 35, including two pairs of right and left curved bodies I, II, III and IV, (with bodies I and II encircling the left pair of pulmonary veins and bodies III and IV encircling the right pair of pulmonary veins), a fifth body V shown extending between the left and right pairs of pulmonary veins, and a sixth body VI shown extending downwardly from the right pair of pulmonary veins to a region in the vicinity of the mitral annulus. One of the bodies I-VI may extends to the vicinity of the left atrial appendage either as an alternative or in addition to the locations shown.

The epicardially-disposed bodies I-VI may be similar to any of the previously described embodiments or a combination thereof. One or more of these bodies I-VI may further include magnets or magnetic material, similar to the bodies 280 and 282 previously described in FIG. 31, which are carried at their respective tips, heels and/or other locations and oriented so as to be attracted to corresponding magnets or magnetic material in an another body to assist placement on the epicardial surface of the heart in addition to other areas.

Introduction of the epicardially-disposed bodies I-VI into the patient's body may be achieved through one or more incisions and may be introduced under fluoroscopic guidance or other like methods. The epicardially-disposed bodies may be introduced virtually simultaneously or, alternately, in series by sequentially inserted one body after another into the patient. Preferably, one flexible, endocardially-disposed body is serially moved into register with each of the approximately 5 or 6 epicardially-disposed bodies for magnetic attraction on opposite sides of the respective tissue layer and the tissue is ablated therebetween, as previously described above in FIGS. 29-30. Then the endocardially-disposed body is released from its magnetic attraction and is moved to each successive epicardially-disposed body where the steps are repeated until ablation is completed at each location.

FIG. 36 shows the apparatus and method employing a guide facility 310 which is shaped or curved to follow the surface of the heart. Several types of guide facilities are possible including but not limited to a wire, a tube, surgical tape, or the like. The guide facility is preferably capable of shape retention and may be manually shaped by the operator in a desired orientation prior to insertion into a patient's body. The guide facility may be introduced into the patient with the aid of a guiding catheter or other conventional methods and may be advanced to the selected tissue location using fluoroscopy or the like.

As shown in FIG. 36, a first or epicardially disposed body, generally indicated at 312, which is similar to the body 280 of FIGS. 29-30 may further cooperatively receive the guide facility 310 to assist in guiding the body to the selected tissue location. In FIG. 36, the epicardially-disposed body 312 includes a channel or lumen 314 which preferably extends from a distal end, generally indicated at 316, of the body to a more proximal location. The guide facility 310 is slidably received within the channel 314 as shown in FIG. 36. The body in FIG. 36 is preferably flexible along at least a portion of its length from its distal end to a more proximal location and may further be flexible along its entire length. FIG. 36 shows the distal end 316 of the epicardially-disposed body 312 flexibly conforming to the shape of the guide facility 310 as the body is slidably advanced along the guide facility. The epicardially-disposed body 312 is advanced distally along the pathway created by the guide facility until it engages the selected tissue location.

FIGS. 37-54 show another embodiment of the apparatus which is adapted for positioning on opposed sides of a layer of cardiac tissue so as to ablate the tissue therebetween. In FIG. 37, the apparatus includes first and second bodies, generally indicated at 312 and 314, respectively. Each of the bodies has corresponding distal ends, 316 and 318, respectively shown in FIG. 37, and an ablation member, which in FIGS. 37A and 37B, is preferably shown as an electrode, respectively at 320 and 322. The electrode may be a coil, flexible strip of conductive metal or any form previously described. The ablation energy source may be supplied by RF energy or any other source as previously described. An electrically insulative material 324 preferably surrounds a portion of each electrode which is not intended for contact with the cardiac tissue.

In FIGS. 37 and 37A-C, the apparatus further includes an elongated housing 326 through which the first and second bodies 312 and 314 extend. The housing 326 may be a catheter, tubular member or other insertion device which facilitates insertion of the first and second bodies 312 and 314 into a patient. Alternatively, the first and second bodies 312 and 314 may be separately inserted each through a separate housing. When the first and second bodies 312 and 314 are placed for ablation on opposite sides of a layer of tissue, they are advanced distally relative to the housing. The proximal ends of each of the first and second bodies may have corresponding tabs 328 and 330 which protrude outwardly through longitudinal slots 332 formed in the walls of the housing, one slot being shown by way of example in FIG. 37C. Other methods may also be used to slidably advance the first and second bodies relative to the housing. FIG. 38 shows the second body after its distal end 318 is advanced distally. In FIG. 39, the distal end 316 of the first body 312 is also advanced in a distal direction. As described in further detail below, the distal ends 316 and 318 engage opposed sides of a layer of tissue.

In FIGS. 37-40, the apparatus further includes a compression sleeve 334 which includes a generally tubular portion that surrounds or encircles the first and second bodies 312 and 314 along a portion of their length. The compression sleeve 334 also includes an extension which protrudes outside of the housing 326 and may be used to facilitate movement of the compression sleeve in a distal direction so as to bias the first and second bodies toward one another, as shown in FIG. 40, and clamp the layer of tissue. A corresponding slot, similar to that shown in FIG. 37C, may be formed in the housing to accommodate slidable movement of the portion of the compression which extends outside of the housing 326.

In FIG. 41 and 42 the apparatus of FIGS. 37-40 preferably includes a piercing element 336 which extends distally from the housing 326. The piercing element 336 may be a needle, wire or other tool which will be apparent to one skilled in the art. The piercing element 336 creates an opening in the layer of tissue which is sufficient for at least one of the first and second bodies 312 and 314 to be inserted, as will be described below. The piercing element 336 is preferably smaller in diameter than either of the first and second bodies 312 and 314 so as to minimize the amount of tissue that is cut. The piercing element 336 may be attached to the distal end of the housing 326 or, alternatively, it may be slidably inserted into the housing and advanced independently of remaining portions of the apparatus. The piercing element 336 may further allow for the injection of a contrast medium into the tissue layer. The contrast medium may assist in location of the selected ablation site. One or more location or visualization devices may be used to locate and view the contrast medium.

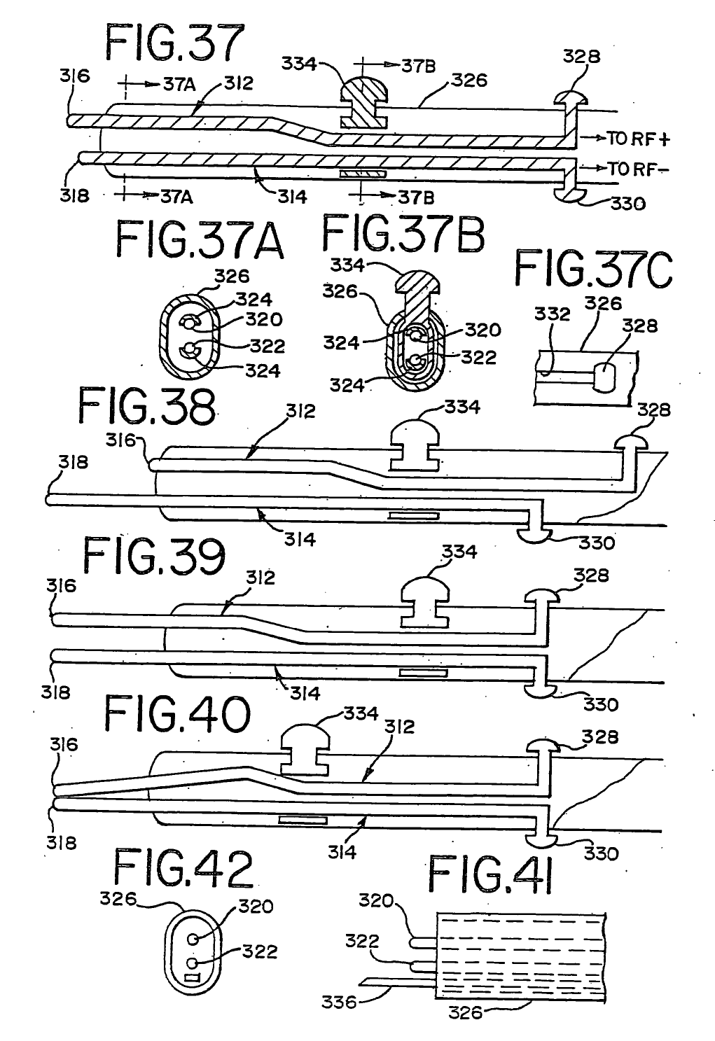

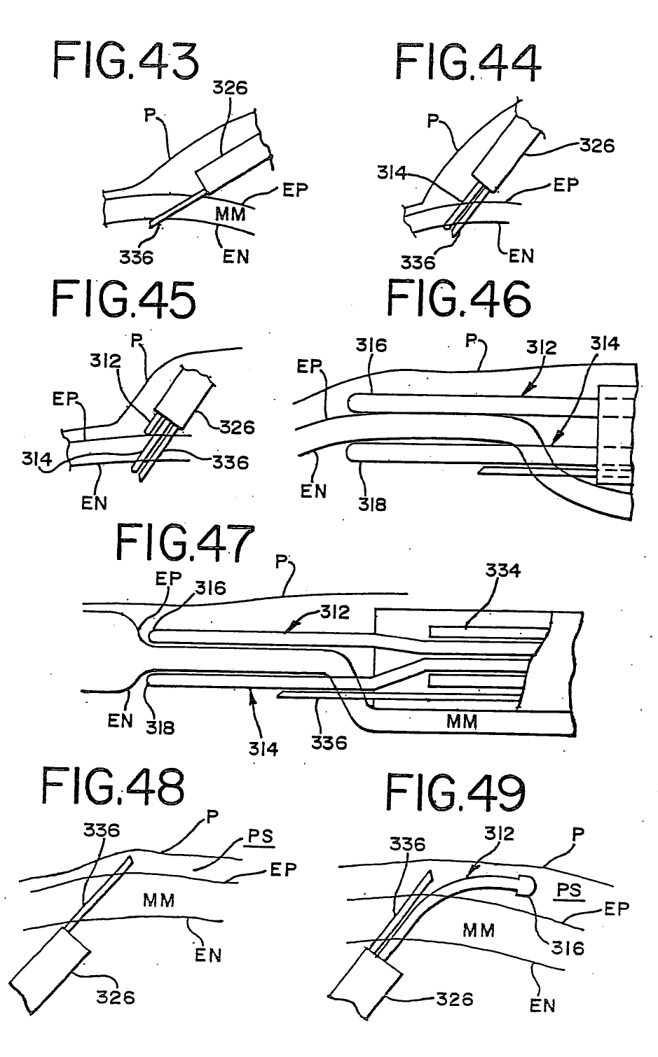

FIGS. 43-47 show one method for ablating opposed sides of cardiac tissue using the apparatus shown in FIGS. 37-42. The apparatus is inserted into the body of a patient through any of the approaches previously described such as sub-xyphoid, intercostal or other approaches. An incision is made into the pericardium by the piercing element 336 or another suitable cutting tool so as to allow insertion of the distal end of the housing 326.

In FIG. 43, the piercing element 336 is advanced and punctures the tissue of the heart in the vicinity of the selected cardiac tissue which has been identified for ablation. For example, the piercing element 336 may penetrate one of the atrial walls. FIG. 43 shows the piercing element 336 piercing the epicardial surface EP of the heart and emerging on the other side of the layer of tissue, i.e., the endocardial surface EN.

In FIG. 44, one of the first and second bodies 312 and 314 is inserted through the opening created by the piercing element 336, the second body 314 being shown by way of example in FIG. 44. The distal ends 316 and 318 of the first and second bodies 312 and 314 preferably have blunt tips so that they do not enlarge the opening any more than necessary to effect the medical procedure. The second body 314 is advanced distally through the opening and engages the endocardial surface EN. In FIG. 45, the first body 312 is also advanced distally except that it engages the epicardial surface EP of the selected tissue layer.

In FIGS. 46-47, the distal ends 316 and 318 of the first and second bodies 312 and 314 are fully advanced and positioned for ablation. The distal ends 316 and 318 are respectively engaged with the epicardial and endocardial surfaces EP and EN. In particular, the respective electrodes 320 and 322 of the distal ends 316 and 318 are in engagement with the opposing surfaces of the layer of tissue. In FIG. 47, the compression sleeve 334 is distally advanced to bias the first and second bodies 312 and 314 towards each other. The layer of tissue is clamped between the distal ends 316 and 318 and activation of the ablation source may commence to effect ablation of the tissue. After ablation, the compression sleeve 33.4 is retracted followed by retraction of the first and second bodies 312 and 314. The apparatus may be repositioned for other ablation sites or removed from the patient's body.

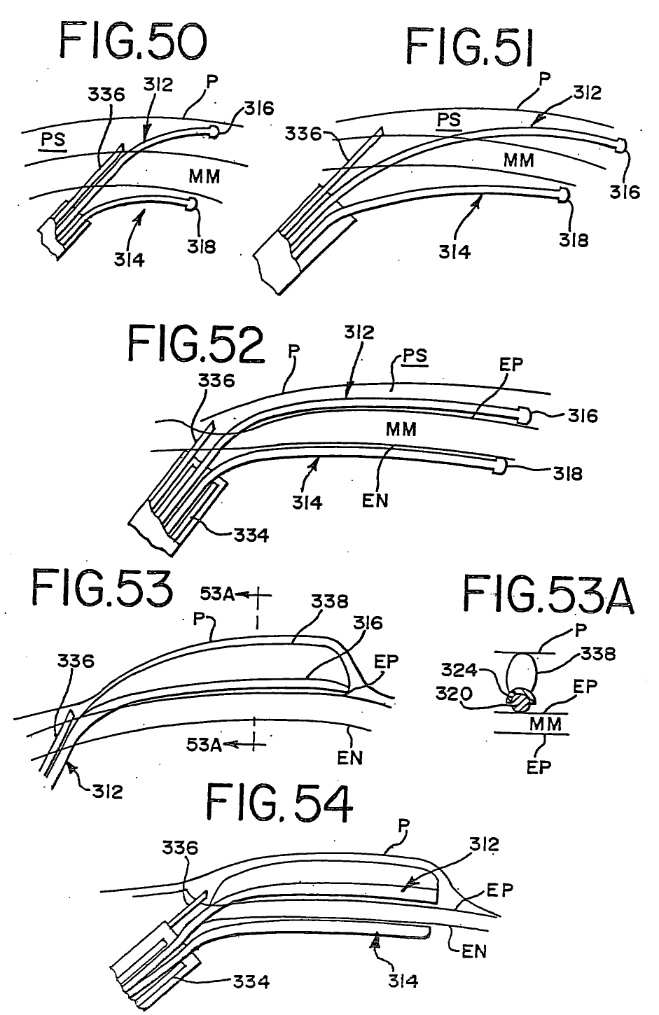

FIGS. 48-52 show another method using the embodiment shown in FIGS. 37-42. In FIG. 48, the apparatus is preferably advanced intravascularly to the heart. The piercing member 336 pierces a heart wall from an interior location and creates an opening, similar to that shown in FIG. 43 which extends between the epicardial and endocardial surfaces EP and EN of the heart. The piercing element 336 preferably does not puncture the pericardium P. In FIG. 49, the distal end 316 of the first body 312 is inserted through the opening created by the piercing element 336 and has a blunt tip so as to avoid piercing the pericardium P. The distal end 316 is advanced into the intrapericardial space PS and engages the epicardial surface EP. In FIG. 50, the distal end 318 of the second body 314 is advanced distally to engage the endocardial surface EN on the opposite side of the layer of tissue. FIG. 52 shows the distal ends 316 and 318 of the corresponding first and second bodies 312 and 314 engaging opposed sides of the cardiac tissue for ablation.

FIGS. 53-54 shows a modified embodiment to that shown in FIGS. 37-42 except that it further includes an expandible member 338 which is carried by the distal end 316 of the first body 312. The expandible member 338 may be comprised of any structure previously shown or described. In FIG. 53A, the expandible member 338 is attached to the first body 312 along the insulated electrode 320 on the side of the electrode which does not engage the epicardial surface EP. The first body 312 is inserted and advanced as previously described above except that the expandible member 338 is unexpanded during insertion of the first body through the myocardium MM, as shown in FIG. 49. Then the expandible member 338 is expanded as shown in FIGS. 53-54. After expansion of the expandible member, the electrode 320 at the distal end 316 is biased into engagement with the epicardial surface EP. Similar to the methods previously described, the compression sleeve 334 is advanced distally to clamp the layer of tissue between the distal ends 316 and 318.

Another advantage of the apparatus is that it can easily be adapted to a minimally invasive approaches such as intercostal, sub-xyphoid or other similar approaches. Each of the first and second bodies in any of the embodiments described above may been reduced to a 5 mm diameter device, and can probably be reduced to 3 mm or less.

Accordingly, an apparatus and method for performing transmural ablation has been provided that meets all the objects of the present invention. While the invention has been described in terms of certain preferred embodiments, there is no intent to limit the invention to the same. Instead it is to be defined by the scope of the appended claims.

1. An apparatus for ablating a layer of tissue having opposed sides comprising:
a first elongated body including a distal end, a proximal end, and a first ablation member, at least a portion of the first body being positioned adjacent one side of the tissue; and
a second elongated body including a distal end, a proximal end, and a second ablation member, at least a portion of the second body being positioned adjacent an opposed side of the tissue, the first and second bodies being positioned in opposed relationship on the opposite sides of the tissue at a selected cardiac location for ablation
at least one of the portions of the first and second bodies having a contoured surface and the other of the first and second bodies having a complementary surface which forms a mating relationship with the contoured surface on opposite sides of the tissue at the selected cardiac location for ablation.

2. The apparatus of claim 1 wherein at least selected one of the first and second bodies includes a source of magnetic force adjacent one side of the tissue and the other of the first and second bodies includes a magnetically attractive element responsive to the magnetic force adjacent the other side of the tissue.

3. The apparatus of claim 1 wherein at least a portion of the first body is positioned on an epicardial surface and at least a portion of the second body is positioned on an endocardial surface.

4. The apparatus of claim 1 further including at least one expandible member disposed on selected one of the first and second bodies.

5. The apparatus of claim 1 further comprising a piercing element which is adapted to extend from the distal end of one of the first and second bodies.

6. The apparatus of claim 1 further comprising a compression sleeve surrounding the first and second bodies which is movable to clamp the layer of tissue between the first and second bodies.

7. A method of ablating a layer of tissue having opposed sides, comprising:
providing a first body including a first ablation member and a source of magnetic force adjacent one side of the tissue, an expandible member located on the first body in the vicinity of the source of magnetic force;
inflating the expandible member to move the source of magnetic force in a direction away from the tissue;
providing a second body including a second ablation member and a magnetically attractive element responsive to the magnetic force adjacent to the other side of the tissue;
deflating the expandible member to move the source of magnetic force in a direction toward the tissue, the magnetic attraction between the source and the attractive element adapted to align the first and second bodies in opposed relationship on the opposed sides of the tissue; and
activating the ablation members at the sides of the tissue layer to ablate the tissue.

8. The method of claim 7 wherein the step of providing a first body includes a second expandible member disposed on the first body in opposed relation to the first named expandible member, and the step of deflating the first named expandible member includes inflating the second expandible member to bias the source of magnetic force adjacent the tissue.

9. The method of claim 7 further including the steps of:
reinflating the first named expandible member to decrease the magnetic attraction between the source of magnetic force of the first body and the magnetically attractive element of the second body to allow repositioning of the second body.

10. The method of claim 9 wherein the step of providing the first body includes a plurality of first bodies, each having an ablation member, a source of magnetic force and an expandible member in the vicinity of the source of magnetic force, each first body being positioned at a different cardiac location selected for ablation adjacent one side of the tissue;
inflating the respective expandible member of the first body to move the respective source of magnetic force in a direction away from the tissue;
positioning the second body adjacent the other side of the tissue in the vicinity of each respective first body;
deflating the respective expandible member of the respective first body; and
repeating the step of activating for each cardiac location.

11. The method of claim 10 wherein the step of providing a first body includes a plurality of bodies which are positioned on an epicardial surface of the heart and the step of providing a second body includes at least one body positioned on an endocardial surface of the heart.

12. The method of claim 11 wherein the step of providing a first body includes approximately six bodies.

13. An apparatus for ablating a layer of tissue having opposed sides comprising:
a first elongated body including a distal end, a proximal end, a first ablation member and a source of magnetic force;
a second elongated body including a distal end, a proximal end, a second ablation member and a magnetically attractive element responsive to the magnetic force, the magnetic attraction between the source and the attractive element adapted to align the first and second bodies in opposed relationship on the opposite sides of the tissue at a selected cardiac location; and
at least one expandible member disposed on selected one of the first and second bodies.

14. The apparatus of claim 13 wherein the expandible member is a balloon.

15. The apparatus of claim 13 wherein a first expandible member is located on the first body and is disposed in the vicinity of the source of magnetic force, the expandible member being inflatable to move the source of magnetic force away from the side of the tissue and being deflatable to move the source of magnetic force toward the side of the tissue.

16. The apparatus of claim 15 wherein a second expandible is located on the first body in opposed relation to the first expandible member, the second expandible member being inflatable to bias the source of magnetic force adjacent the side of tissue.

17. The apparatus of claim 16 wherein each of the first and second expandible members is connected to a inflation lumen which extends proximally to a fluid source located outside a patient's body.

18. The apparatus of claim 16 wherein each of the first and second expandible members extends from the distal end of the first body to a more proximal location which is in the vicinity of a proximal edge of the source of magnetic source.

19. The apparatus of claim 13 wherein the first body engages an epicardial surface of the heart and the second body engages an endocardial surface of the heart.

20. The apparatus of claim 19 wherein a plurality of first bodies are each positioned at a different location on the epicardial surface of the heart, each first body having an ablation member and a source of magnetic force, the respective sources of magnetic force being magnetically attracted to each other across pericardial reflections of the heart.
